(19) United States
(12) Patent Application Publication
(10) Pub. No.: US 2024/0181070 A1
(43) Pub. Date: Jun. 6, 2024

(54) NANOVACCINES FOR TREATMENT OF VIRAL DISEASES

(71) Applicant: Ramot at Tel-Aviv University Ltd., Tel-Aviv (IL)

(72) Inventors: Ronit SATCHI-FAINARO, Tel Aviv (IL); Helena FLORINDO, Tel Aviv (IL)

(73) Assignee: Ramot at Tel-Aviv University Ltd., Tel-Aviv (IL)

(21) Appl. No.: 18/285,394

(22) PCT Filed: Apr. 7, 2022
(86) PCT No.: PCT/IL2022/050366

§ 371 Date: Oct. 3, 2023

Related U.S. Application Data
(60) Provisional application No. 63/172,144, filed on Apr. 8, 2021.

(51) Int. Cl. A61K 47/64; A61K 39/00; A61K 39/215; A61K 47/69

(52) U.S. Cl. CPC A61K 47/646; A61K 39/215; A61K 47/6455; A61K 47/6937; A61K 2039/6093

(57) ABSTRACT

A polymeric nanoparticle is disclosed which comprises: (i) at least one SARS-CoV-2 derived antigen which is capable of producing a T-cell and/or B cell response: and (ii) an antigen presenting cell targeting moiety which is attached to the outer surface of the nanoparticle. Use of the nanoparticle for treating COVID-19 is also disclosed.

### RELATED APPLICATION/S

This application claims the benefit of priority of U.S. Patent Application No. 63/172,144 filed 8 Apr., 2021, the contents of which are incorporated herein by reference in their entirety.

### SEQUENCE LISTING STATEMENT

The ASCII file, entitled 91066 Sequence Listing.txt, created on 7 Apr. 2022, comprising 25,630 bytes, submitted concurrently with the filing of this application is incorporated herein by reference.

### FIELD AND BACKGROUND OF THE INVENTION

The present invention, in some embodiments thereof, relates to polymeric nano-vaccines and, more particularly, but not exclusively, to their use in treating or preventing coronaviral diseases, such as COVID-19.

As of March 2022, the coronavirus disease 2019 (COVID-19) pandemic, caused by severe acute respiratory syndrome coronavirus 2 (SARS-CoV-2), continues to have tremendous impact on global economy, education, social relations and human health. Concerted efforts involving research centers, hospitals, pharmaceutical and biotechnology companies, and regulatory agencies accelerated the development and approval for human use of several vaccines at an unprecedented pace. Current SARS-CoV-2 vaccines that are authorized or received approval for emergency use are based on distinct technologies: adenoviral vector (Johnson & Johnson's Janssen, Oxford-AstraZeneca and Sputnik V), mRNA (Pfizer-BioNTech and Moderna), protein-based (Novavax) and inactivated whole-virus vaccines (Sinopharm and Sinovac). Nanotechnology-based platforms, which were already used in ˜550M vaccination doses globally, have played a paramount role in supporting this rapid progress in vaccination against SARS-CoV-2 infection, by enabling a prompt scale-up production and commercial manufacturing of an immunogenic synthetic vaccine. So far, all nanoplatforms approved as SARS-CoV-2 vaccines by the European medicine agency (EMA) and the food and drug administration (FDA) incorporate mRNA to enable Spike protein production by host cells. These nanotechnology-based vaccines hold several advantages over traditional vaccination approaches. However, they also pose several challenges such as the need for low temperature storage enabling a reasonable shelf-life, in addition to the high costs associated with manufacturing, handling, and storage conditions. Most of the COVID-19 vaccines in use indeed require a controlled temperature storage between 2-8° C. (inactivated SARS-CoV-2, adenovirus-based and protein-based vaccines) or −18° C. to −90° C. (mRNA vaccines, viral vector).

Although ˜65% of the world's population have already received at least one dose of a COVID-19 vaccine, the global vaccination rates are still scarce, as less than 5% of the population in low-income countries are fully vaccinated. The inefficient immunization supply chains (production, distribution, administration), healthcare systems or logistics (e.g., need for cold chain storage) have contributed to this inequitable allocation of vaccines in developing countries. These unbalanced regional vaccination rates led to recurrent outbreaks, as SARS-CoV-2 Omicron variant (B.1.1.529) the novel variant of concern. Therefore, to overcome this challenge, it is critical to develop effective vaccines based on technologies with long shelf-live at room temperature, such as lyophilized vaccines.

The current knowledge on COVID-19 indicates that a natural protective immunity against SARS-CoV-2 infection is unlikely. Although, the current vaccines have been instrumental to reduce disease burden and control the pandemic to some extent, it is still unclear what is the duration of the long-term immunity achieved by them, their ability to prevent virus transmission or protect against emerging variants. Peptide vaccines constitute an alternative that solves some of these major challenges. These vaccines present a relatively simple, reliable and cost-effective manufacturing process that leads to a stable product. In fact, these vaccines are stable in their lyophilized form at room temperature, which makes the temperature control redundant. Moreover, the delivery of peptides as epitopes by nanoparticles (NP) trafficking to distinct intracellular pathways within antigen-presenting cells (APC), overcomes the need for protein translation, folding and processing of mRNA vaccines. The delivery of loaded antigens to the site of conjugation to Major Histocompatibility Complex (MHC) molecules leads to extensive antigen presentation enabling the induction of a broad spectrum immunity, crucial to overcome infection by different SARS-CoV-2 variants.

Background art include US Patent Application No. 20100278919, US Patent Application No. 20180200196, US Patent Application No. 20160051698, US Patent Application No. 20170042994, International Patent Application WO 2018/039332, Gutjahr et al., Vaccines 2016, 4, 34; doi:10.3390 and Keselowsky et al., Human Vaccines 7:1, pages 37-44; January 2011 and PCT/IL2019/051420.

### SUMMARY OF THE INVENTION

According to an aspect of the present invention, there is provided a polymeric nanoparticle comprising:
(i) at least one SARS-CoV-2 derived antigen which is capable of producing a B-cell and/or a T-cell response; and
(ii) an antigen presenting cell targeting moiety which is attached to the outer surface of the nanoparticle.

According to an aspect of the present invention, there is provided a vaccine comprising the polymeric nanoparticle described herein.

According to an aspect of the present invention, there is provided a method of treating or preventing COVID-19 in a subject in need thereof comprising administering to the subject a therapeutically effective amount of the polymeric nanoparticles of any one of claims 1-43, thereby treating or preventing COVID-19.

According to a particular embodiment, the antigen presenting cell is a dendritic cell.

According to a particular embodiment, the polymeric nanoparticle further comprises a polynucleotide agent capable of downregulating an amount of a polypeptide in the dendritic cell, wherein the polynucleotide agent is entrapped in the nanoparticle.

According to a particular embodiment, the polymeric nanoparticle further comprises d-α-tocopheryl polyethylene glycol 1000 succinate (TPGS).

According to a particular embodiment, the polymeric nanoparticle further comprising at least one toll-like receptor ligand which is entrapped in the nanoparticle.

According to a particular embodiment, the polymeric nanoparticle further comprises at least one retinoic-acid-inducible protein 1 (RIG-I)-like receptor ligand which is entrapped in the nanoparticle.

According to a particular embodiment, the polymeric nanoparticle at least one adjuvant.

According to a particular embodiment, the polymeric nanoparticle is preferentially endocytosed by dendritic cells as compared to macrophages.

According to a particular embodiment, the at least one SARS-CoV-2 derived antigen comprises at least two SARS-CoV-2 derived antigens.

According to a particular embodiment, the first SARS-CoV-2 derived antigen is a MHC class I T cell epitope and the second SARS-CoV-2 derived antigen is a MHC class II T cell epitope.

According to a particular embodiment, the at least one SARS-CoV-2 derived antigen is selected from the group consisting of a B cell epitope, an MHC class I T cell epitope and an MHC class II T cell epitope.

According to a particular embodiment, the MHC class II T cell epitope is a B cell epitope.

According to a particular embodiment, the at least one SARS-CoV-2 derived antigen is derived from the spike protein of the SARS-CoV-2.

According to a particular embodiment, the at least one SARS-CoV-2 derived antigen is derived from the nucleocapside protein of the SARS-CoV-2.

According to a particular embodiment, the at least one SARS-CoV-2 derived antigen is derived from the membrane protein of the SARS-CoV-2.

According to a particular embodiment, the at least one SARS-CoV-2 derived antigen is derived from the envelope protein of the SARS-CoV-2.

According to a particular embodiment, the at least one SARS-CoV-2 derived antigen is derived from a non-structural protein of the SARS-CoV-2.

According to a particular embodiment, the at least one SARS-CoV-2 derived antigen is derived from an open reading frame (ORF) protein of the SARS-CoV-2.

According to a particular embodiment, the at least one SARS-CoV-2 derived antigen comprises the amino acid sequence selected from the group consisting of SEQ ID NOs: 1-142.

According to a particular embodiment, the at least one SARS-CoV-2 derived antigen comprises the amino acid sequence selected from the group consisting of SEQ ID NOs: 1-21.

According to a particular embodiment, the polymeric particle the at least one SARS-CoV-2 derived antigen comprises the amino acid sequence as set forth in SEQ ID NO: 14 and/or SEQ ID NO: 15.

According to a particular embodiment, the polymeric particle has a diameter between 100-300 nm, as measured by dynamic light scattering.

According to a particular embodiment, the polymeric particle has a diameter no greater than 300 nm, as measured by dynamic light scattering.

According to a particular embodiment, the polymeric particle has an average diameter of about 200 nm, as measured by dynamic light scattering.

According to a particular embodiment, the particle is fabricated from at least one polymer selected from the group consisting of poly(lactic-co-glycolic acid) (PLGA), Polyethylene glycol-PLGA (PEG-PLGA), Poly(lactic acid) (PLA), PEG-PLA, Polycaprolactone (PCL) and PEG-PCL.

According to a particular embodiment, the particle is fabricated from PLGA and PLA.

According to a particular embodiment, the particle further comprises PVA.

According to a particular embodiment, the ratio of PLGA:PLA is 1:4.

According to a particular embodiment, the at least one adjuvant comprises a Toll-like receptor (TLR) ligand.

According to a particular embodiment, the Toll-like receptor (TLR) ligand is selected from the group consisting of a ligand of TLR2, a ligand of TLR3, a ligand of TLR4, a ligand of TLR5, a ligand of TLR7/8 and a ligand of TLR9.

According to a particular embodiment, the ligand is selected from the group consisting of zymosan, Polyinosinic-polycytidylic acid (Poly(I:C)), Monophosphoryl Lipid A (MPLA)), glucopyranosyl lipid adjuvant (GLA), flagellin, Gardiquimod, Imiquimod (R837) Resiquimod. Inducible T-cell co-stimulator ligand (ICOSL) and CpG oligodeoxynucleotides (CpG ODN).

According to a particular embodiment, the at least one adjuvant comprises a retinoic-acid-inducible protein 1 (RIG-I)-like receptor ligand.

According to a particular embodiment, the (RIG-I)-like receptor ligand is selected from the group consisting of a ligand of RIG-I and MDA-5 (melanoma-differentiation-associated gene 5, or Ifih1 or Helicard).

According to a particular embodiment, the ligand is selected from the group consisting of 5′ppp-dsRNA, 3p-hpRNA, Polyinosinic-polycytidylic acid (Poly(I:C)), Poly(dA:dT).

According to a particular embodiment, the at least one adjuvant is selected from the group consisting of hyaluronic acid (HA), poloxamer 407, 2′,3′-cGAMP, chitosan, Dectin-1 agonist laminarin and β-glucan.

According to a particular embodiment, the polymeric nanoparticle further comprises a surfactant.

According to a particular embodiment, the surfactant is selected from the group consisting of d-α-tocopheryl polyethylene glycol 1000 succinate (TPGS), poly(vinyl alcohol) (PVA) and poloxamer 407.

According to a particular embodiment, the polymeric nanoparticle further comprises a polynucleotide agent capable of downregulating an amount of a polypeptide in the dendritic cell.

According to a particular embodiment, the polynucleotide agent is selected from the group consisting of an antisense polynucleotide, siRNA, gRNA, miRNA, a DNAzyme and a Ribozyme.

According to a particular embodiment, the polypeptide is selected from the group consisting of SARS-CoV-2 Spike glycoprotein, Membrane glycoproteins (region 220-241), Nucleocapsid and envelope proteins, SARS Replicase and RNA Polymerase region, TGF-β, VEGFA, PD-L1/PD-1, VEGFR1, VEGFR2, VEGFR3, IDO, RANKL, IL-10, IL-6/IL-6R, IL-1, IL-28A, IL-28B, IL-29, IP-10/CXCL10 (interferon γ-inducible protein 10), CD16, ITAM (immunoreceptor tyrosine-based activation motif), DC-SIGN (dendritic cell specific intercellular adhesion molecule-grabbing nonintegrin), ICAM-3 (intercellular adhesion molecule 3) and PGE2 receptor.

According to a particular embodiment, the polynucleotide agent is siRNA.

According to a particular embodiment, the siRNA is complexed with a polymer.

According to a particular embodiment, the polymer is selected from the group consisting of glutamate chitosan, poly-arginine, alkylated poly(α)glutamate amine (APA) and poly-(α)glutamic acid (PGA).

According to a particular embodiment, the antigen presenting cell targeting moiety is selected from the group consisting of mannose, tri-mannose, PEG-mannose, laminarin and PEG-laminarin.

According to a particular embodiment, the polymeric nanoparticle is for use in treating or preventing COVID-19.

According to a particular embodiment, the administering is subcutaneous, intradermal, intramuscular, intratumoral, intravenous or mucosal.

According to a particular embodiment, the mucosal is nasal, oral, sublingual, ocular, vaginal or rectal.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-L. NP physicochemical characterization. A, Schematic representation of the NV. B. Atomic force microscopy (AFM) images show the spherical shape of NP with a slight roughness surface, scale bar=200 nm. C, Representative image of spherical empty NP by transmission electron microscopy (TEM), scale bar=200 nm. D, Representative image of spherical empty NP by scanning electron microscopy (SEM), scale bar=1 μm. (E-F) hydrodynamic diameter measurements of empty NP and NV (NP that encapsulate the peptides and adjuvants) were collected over time by DLS. e, Lyophilized NP stored in 24° C. f, NP and NV in suspension were stored at two temperatures, 4° C. and 24° C. Data represent mean±s.d. (n=5). Data represent mean±s.d. (n=3). G, ImageStream image of bone marrow DC (BMDC), 1 h incubation with Cy5-labeled empty NP, scale bar=10 μm. H, Cy5-labeled empty NP internalization by BMDC determined by flow cytometry. Non-labeled NP were used as negative controls. α-mannose receptor (MR) antibody (Ab) was introduced to the cells, 1 h before NP incubation. Data represent mean±s.d. (representative graph of 2 independent experiments that demonstrated the same trend). I, Cell viability of BMDC after incubation with NP, for 44 h by XTT. Data represent mean±s.d. (representative graph of 2 independent experiments that demonstrated the same trend). J, In vivo safety timeline, C57BL/6J mice. Assessment of motor function in K, RotaRod test. The motor learning of C57BL/6 male mice was analyzed in a five-lane accelerating RotaRod. L, open field test. The distance traveled by C57BL/6 male mice during a 15-min video recording was analyzed using EthoVision 13XT software. Data represent mean±s.e.m., N=10 animals. All comparisons between the groups were non-significant (NS).

FIGS. 2A-G. Activation of cellular and humoral immunities by COVID-19 NV-7. A, Immunization scheme of C57BL/6J mice. B, Body weight change of C57BL/6J mice, N=10, mean±s.e.m. C-E, T-cell and B-cell activation markers detected by flow cytometry. c, Activation of CD8⁺ and CD4⁺ T cells isolated from spleen. Top left, CD3⁺CD4⁺IL-6⁺. Top right, CD3⁺CD4⁺ IL-10⁺. Bottom left, CD3⁺CD4⁺IL-2⁺. Bottom right, CD3⁺CD8⁺PD-1⁺. D, CD4⁺ T regulatory cells isolated from inguinal lymph nodes of immunized mice. Left, CD4⁺FOXP3⁺CD25⁺. Right, CD4⁺FOXP3⁺PD-1⁺CXCR5⁺. E, Activation of B cell populations isolated from Inguinal lymph nodes. Left, B220⁺CD138⁺. Right, B220⁺ IgG⁺. The graphs represent mean±s.e.m., N=4, statistical analysis: Unpaired t test. F. Mice sera were collected on days 0, 7, 14, 21, 28 and 47 and tested for IgM antibodies (Ab) secretion against SARS-CoV-2 specific antigens by ELISA. The graph shows mean±s.e.m., N=10. F-G, IgM (F) and IgG (G) Ab binding ability was tested against the named peptides. Statistical analysis: repeated measures two-way ANOVA.

FIGS. 3A-C. In vivo screening of COVID-19 vaccine candidates 8-11. A, Immunization scheme of C57BL/6J mice timeline. B, Frequencies of antigen-specific CD4⁺T cells producing T helper 1 (T_{H}1) cytokines interferon-gamma (IFN-γ), tumor necrosis factor-alpha (TNF-α), and IL-2 evaluated 1 week after the second NV dose (day 28) and after stimulation with the relevant SARS-CoV-2 peptides. Data represent mean±s.d., N=3 animals, one-way ANOVA followed by Tukey's multiple comparisons test. C, SARS-CoV-2 peptide-specific IgG and IgM antibodies from blood serum collected on day 35 determined by ELISA. Data represent mean±s.d., N=5 animals selected/group, unpaired Student's t test.

FIGS. 4A-G. NV elicited robust RDB-specific T- and B-cell responses. A, Immunization scheme of C57BL/6J mice timeline. B, cellular response. Frequencies of antigen-specific CD4⁺T cells producing Th1 cytokines, such as IFN-γ, TNF-α, and IL-2; and for Th2 cytokines: IL-4, IL-6 and IL-10 were evaluated 1 week after the second vaccination (day 28) and after splenocyte stimulation with the relevant peptides. Cytokine production (IFN-γ, TNF-α and IL-2) was also evaluated in CD8⁺ T cells. Data represent mean±s.d., N=5 animals, one-way ANOVA followed by Tukey's multiple comparisons test. C, ELISpot representative images of IFN-γ spot forming cells among splenocytes after ex vivo restimulation with peptides P14 and P15 on day 28. Each condition was repeated five times. D, SARS-CoV-2 peptides (P14 and P15) and RBD-specific IgG antibody determined by ELISA. Data represent mean±s.d., N=5-10 animals/group, unpaired Student's t test. E, Challenge assay scheme of C57BL/6J mice timeline. F, SARS-CoV-2 peptide specific IgG antibody determined by ELISA (Number of animals/group: PBS=7, Free=10, Empty NP=9, Empty NP+Adj=8, NV=9, mean±s.e.m.). Statistical analysis was performed using two wat ANOVA (Tukey's multiple comparisons test). G, cellular response. Frequencies of effector memory T cells (CD4⁺ and CD8⁺) and memory B cells were evaluated 1 week after the challenge treatment (day 101). (N=8-10 animals/group±s.e.m.).

FIGS. 5A-I. Co-delivery of NV-8 and siRNA against PD-L1 immunosuppressive pathway increase NV neutralizing-antibody responses. A, PD-L1 and PD-1 mRNA levels from spleen, 55 h post siNC (negative control of scramble siRNA) NV or siPD-L1 NV immunization. mRNA levels measured by qRT-PCR. Data represent mean±s.e.m., N=3-5 animals, unpaired t-test. B, Immunization scheme of C57BL/6J mice timeline. C, PD-L1 and PD-1 mRNA levels, from spleens, on day 28. mRNA levels measured by qRT-PCR. Data represent mean±s.d., N=5 animals, one-way ANOVA followed by Dunnett's multiple comparisons test. D, Germinal center (GC) B cells detected by flow cytometry on day 28. Data represent mean±s.d., N=5 animals, one-way ANOVA followed by Tukey's multiple comparisons test. E, Mouse sera were collected on day 35 and tested for SARS-CoV-2 peptides (P14 and P15)-, RBD-specific IgG, and F, SARS-CoV-2 peptide P15-specific IgG1 and IgG2a antibodies. Data represent mean±s.d., N=5 animals/group, unpaired Student's t test. G, SARS-CoV-2 peptides, and RBD-specific IgG antibody titers determined by ELISA. Data represent mean±s.d., N=5 animals/group, unpaired Student's t test. H, schematic overview of the surrogate virus neutralization test (sVNT). I, neutralizing antibodies determined by sVNT for RBD wild type (WT) and variants (alfa α, beta β, delta Δ, gamma γ, and omicron o). Data represent mean±SD, N=5 animals selected/group, one-way ANOVA followed by Tukey's multiple comparisons test.

FIGS. 6A-C. Intranasal NV-booster elicited a robust T cell response. A, Immunization scheme of C57BL/6J mice timeline. B, cellular response. Frequencies of antigen-specific CD4⁺ T cells producing Th1 cytokines, such as IFN-γ and TNF-α, were evaluated 1 week after the intranasal booster (day 28) and after stimulation with the relevant peptides. Cytokine production (IFN-γ and TNF-α) was also evaluated on CD8⁺ T cells (N=5 animals selected/group±SD). C, Secretory IgA antibodies in the Bronchoalveolar lavage Fluid (BALF) determined by ELISA (N=4 or 5 animals selected/group±SD). IgA antibody levels was also determined in the serum of animals immunized subcutaneously (s.c.) (prime and boost doses). Statistical analysis was performed using one-way ANOVA.

### DESCRIPTION OF SPECIFIC EMBODIMENTS OF THE INVENTION

The present invention, in some embodiments thereof, relates to polymeric nano-vaccines and, more particularly, but not exclusively, to their use in treating or preventing COVID19.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

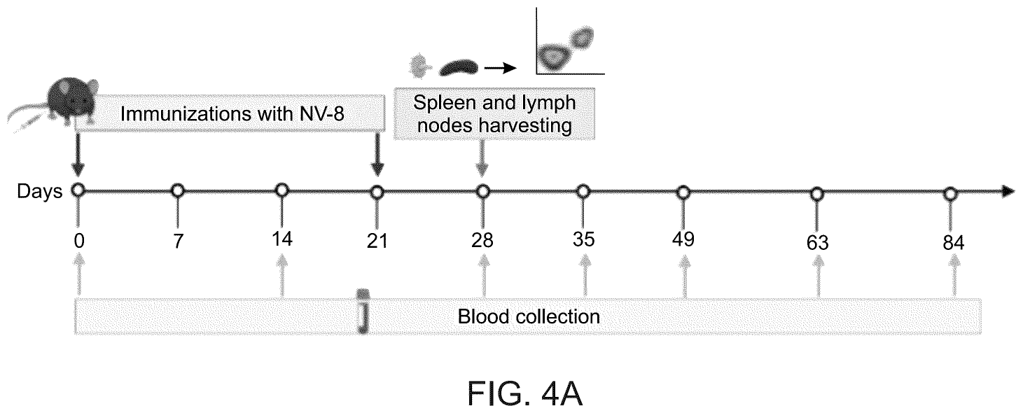

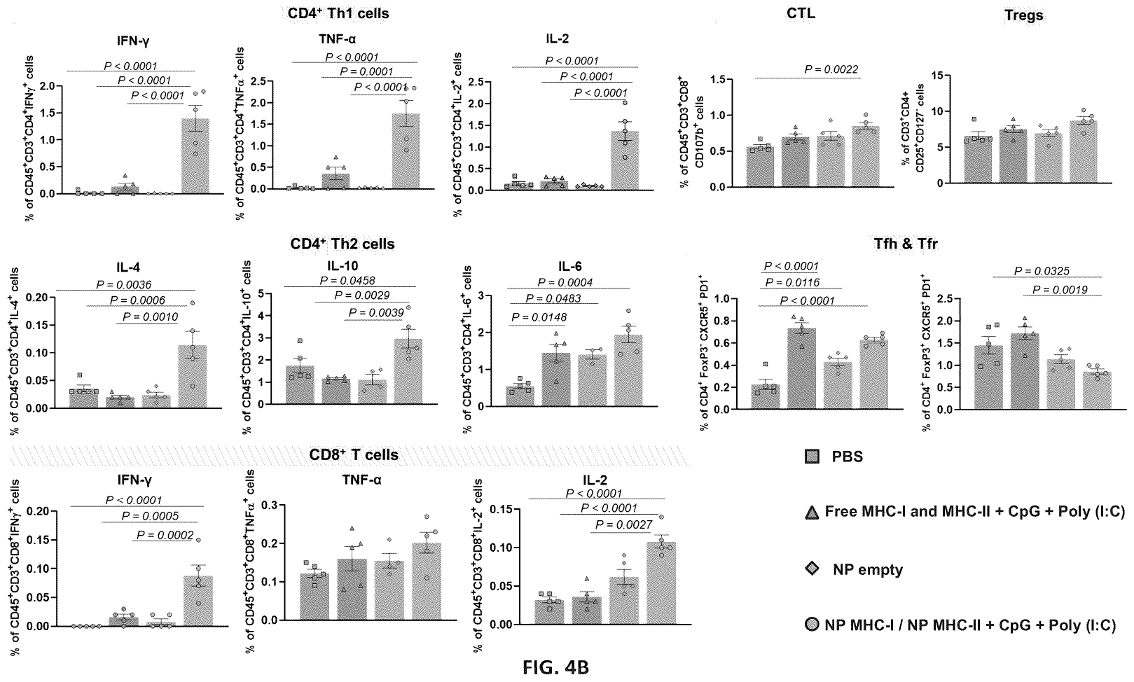

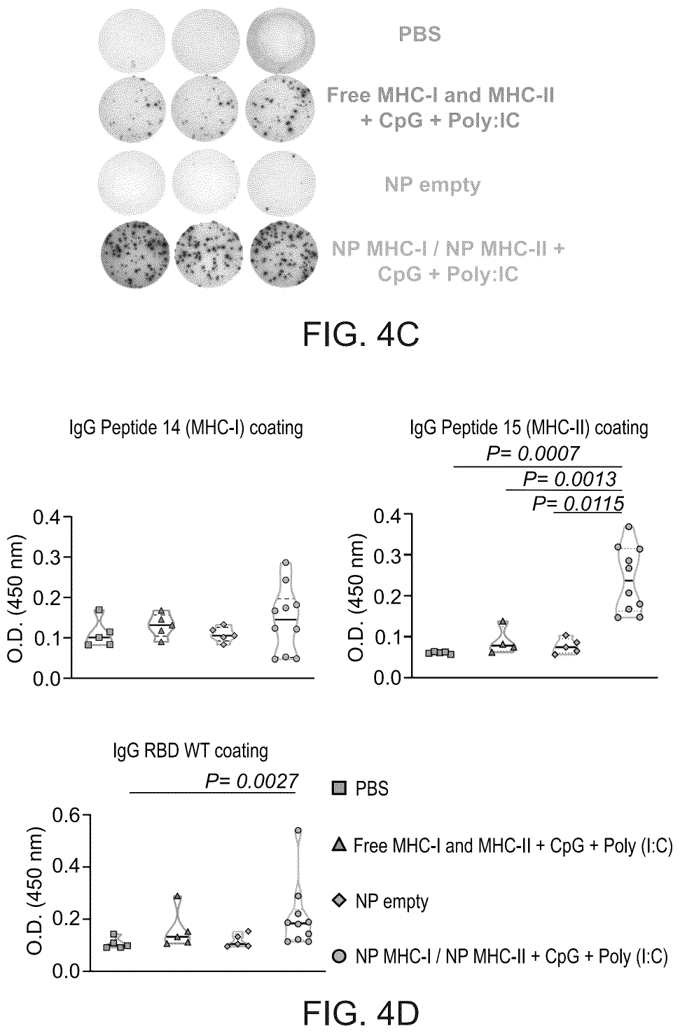

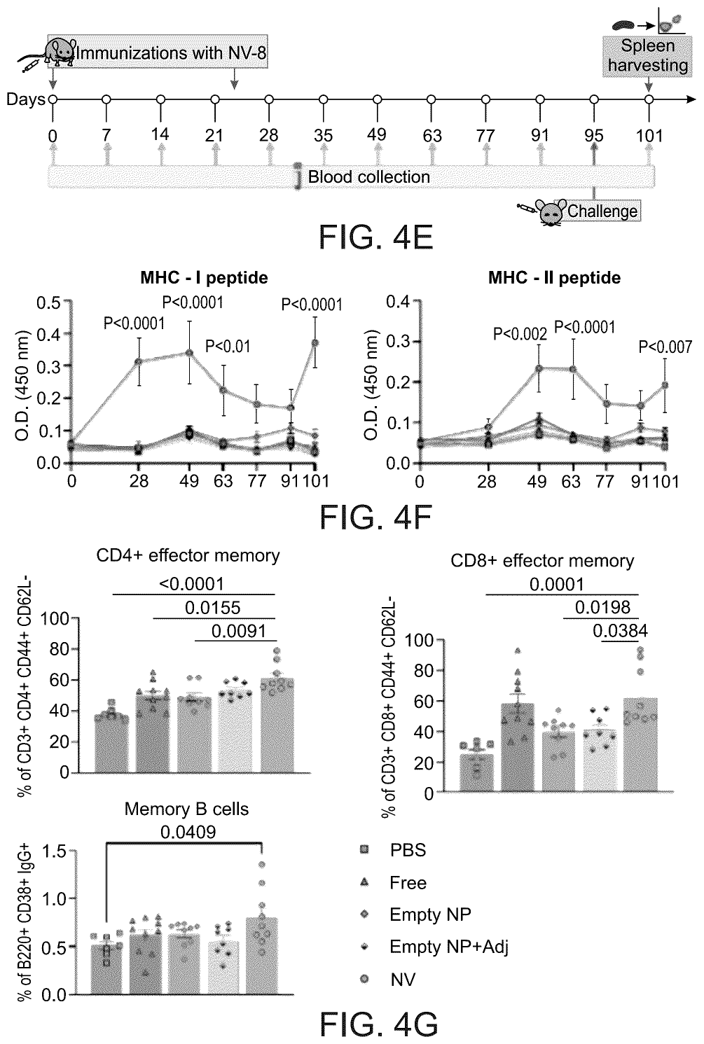

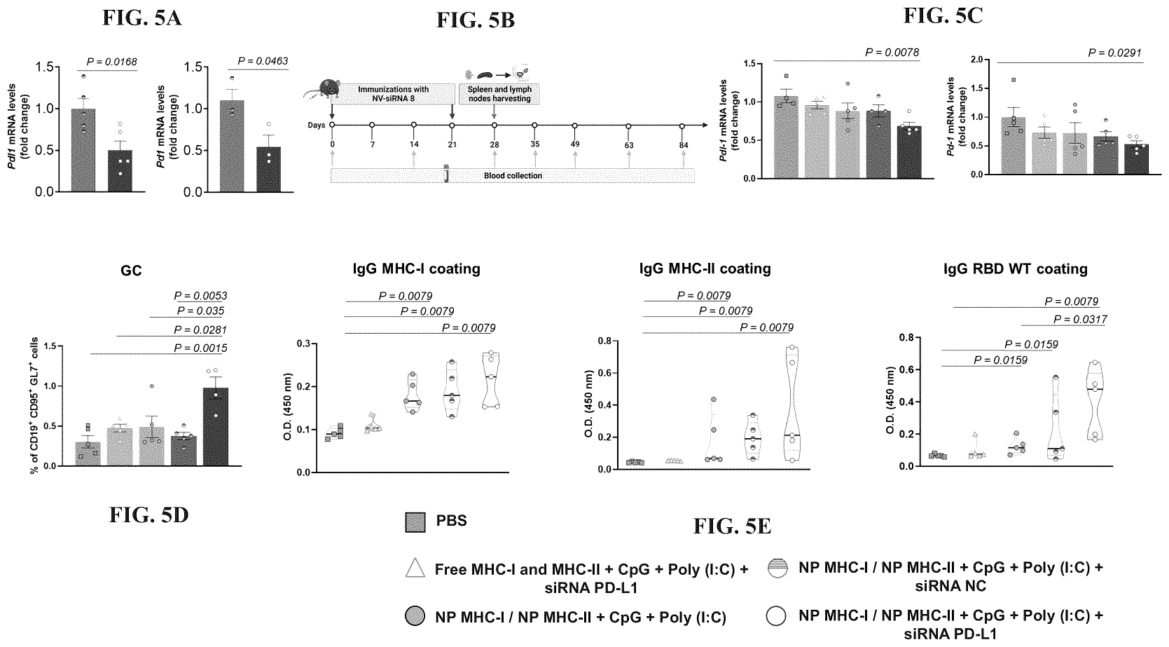

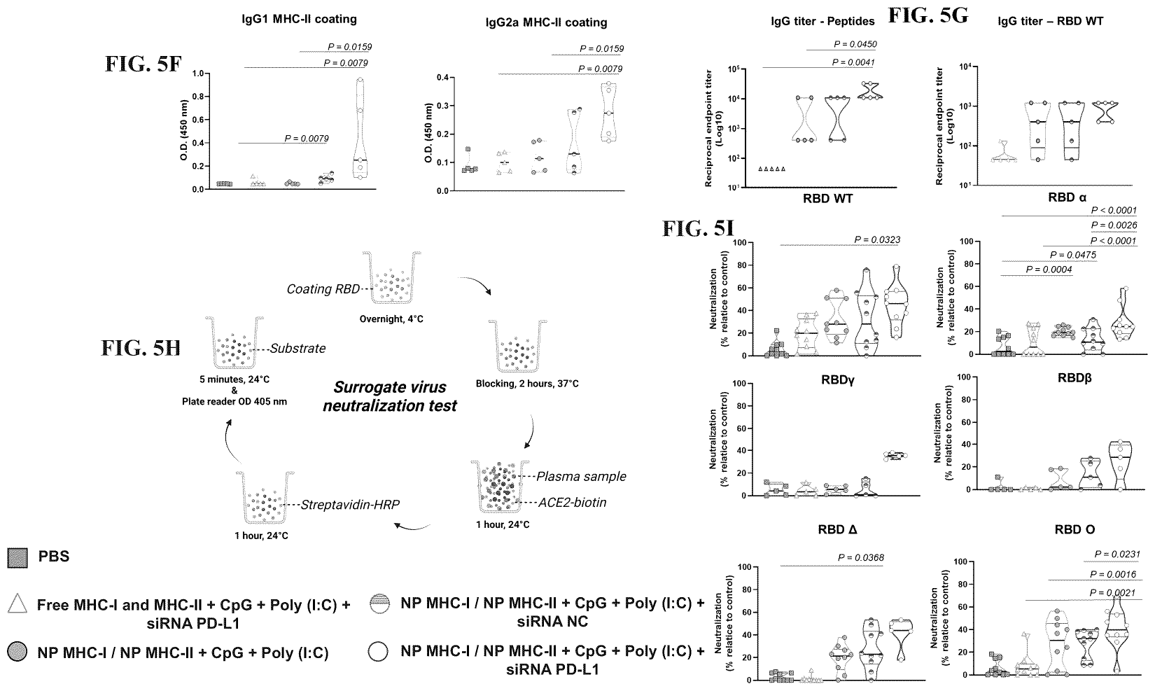

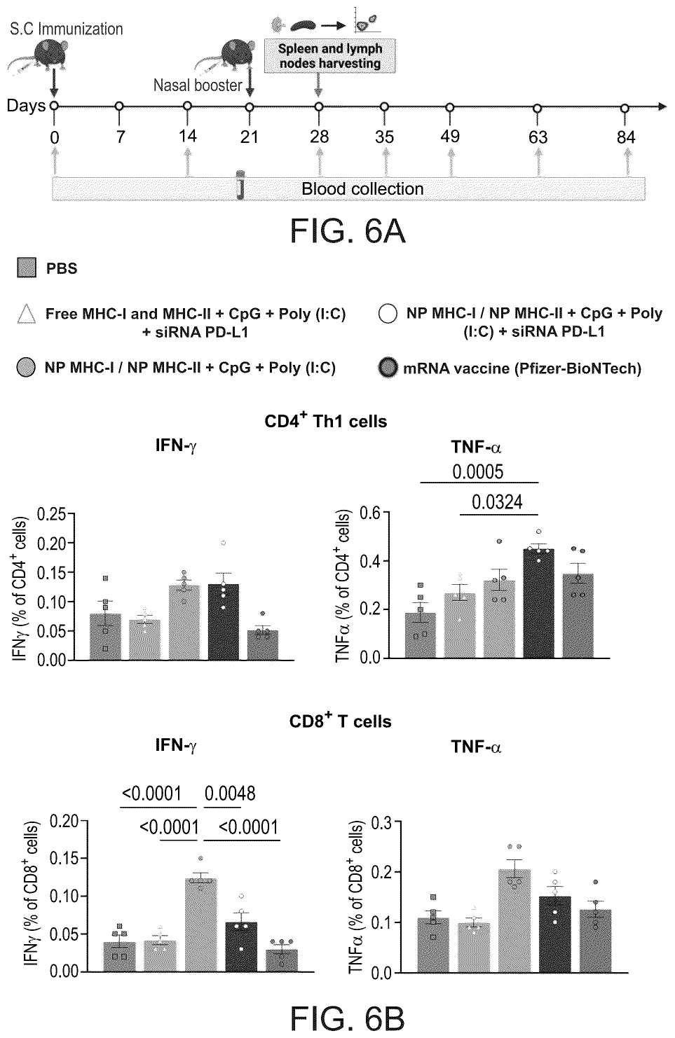

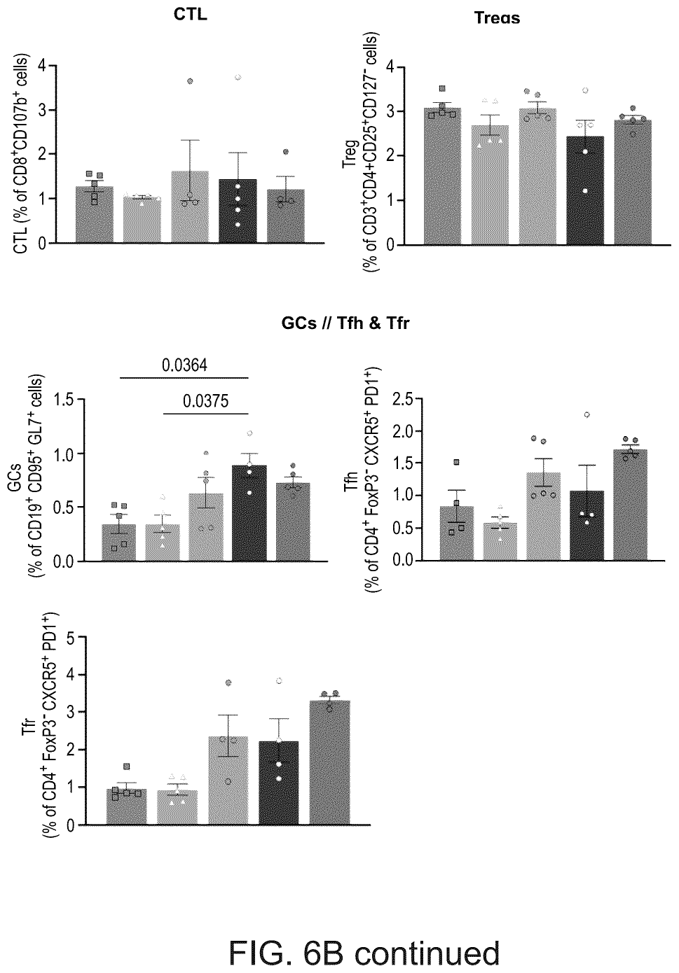

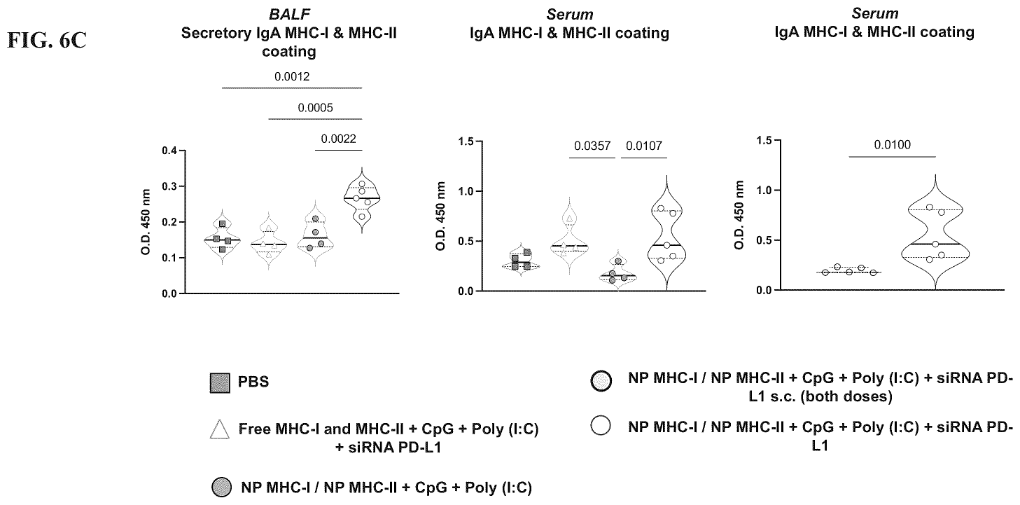

Coronavirus disease 2019 (COVID-19), caused by severe acute respiratory syndrome coronavirus 2 (SARS-CoV-2), has grown into a global pandemic causing tremendous effects on all life aspects beyond morbidity and mortality worldwide. Nanotechnology-based vaccines were among the first to receive the conditional approval for human use, which life-saving impact was possible due to their rapid production and modularity. Unfortunately, highly infectious SARS-CoV-2-mutated variants were able to evade neutralizing antibodies, stressing the global demand for novel vaccines. The present inventors have now developed a next-generation multi-epitope nanotechnology-based vaccine against COVID-19, which co-delivers SARS-CoV-2 peptide antigens, adjuvants, and regulators of the expression of the immune checkpoint PD-L1 into dendritic cells. This nanovaccine enabled the coordinated regulation of PD-L1/PD-1 pathway and multivalent peptide presentation by resident and migratory dendritic cells. Moreover, its long-term stability at room temperature makes it a relevant candidate, especially in developing countries, crucial to prevent the emergence of new SARS-CoV-2 variants. This subcutaneously- or intranasally-administered nanovaccine was able to induce effector CD4 and CD8 T-cell responses (FIG. 4G, FIG. 6B), while triggering a broad humoral immunity capable of neutralizing all five variants of the receptor-binding domain (RBD) of the SARS-CoV-2 spike protein (FIG. 5G and FIG. 5I), in addition to the secretion of IgA at the pulmonary mucosa (FIG. 6C).

Thus, according to an aspect of the present invention there is provided a polymeric nanoparticle comprising:
(i) at least one SARS-CoV-2 derived antigen which is capable of producing a B-cell and/or a T-cell response; and
(ii) an antigen presenting cell targeting moiety which is attached to the outer surface of the nanoparticle.

As used herein, the term “nanoparticle” refers to a particle in the range between 10 nm to 1000 nm in diameter, wherein the diameter refers to the diameter of a perfect sphere having the same volume as the particle.

In some cases, the diameter of the particle is in the range of about 1-1000 nm, 10-500 nm, 20-300 nm, or 100-300 nm, as measured by dynamic light scattering. In various embodiments, the diameter is between 150-200 nm.

In some cases, a population of particles may be present. As used herein, the diameter of the nanoparticles is an average of a distribution in a particular population.

The population of nanoparticles preferably have an average diameter no greater than 300 nm, and even no greater than 200 nm (e.g. 190 nm).

Examples of polymers that may be used to fabricate the nanoparticles of this aspect of the present invention include but are not limited to vinyl polymers, such as polyvinyl aromatics (e.g., polystyrene), polyacrylates (e.g., polymethyl acrylate, polyethyl acrylate), polymethacrylates (e.g., polymethyl methacrylate), polycyanoacrylates, polyacrylonitrile, polyvinyl halides (e.g., polyvinyl chloride, polyvinylidene fluoride, polyvinylidene chloride and/or polytetrafluoroethylene), polyvinyl ketones, polyvinyl ethers (e.g., polyvinyl methyl ether), polyvinyl esters (e.g., polyvinyl acetate) and/or polyvinyl alcohol; polyphosphoesters (e.g., poly[1,4-bis(hydroxyethyl)terephthalate-co-ethyloxyphosphate]); polyurethanes; polyphosphazenes; polyesters, such as polycaprolactone, polylactic acid (e.g., poly(L-lactic acid), poly(D-lactic acid) and/or poly(D,L-lactic acid), polyglycolic acid, polyhydroxybutyrate, polyhydroxyvalerate, polyalkylene succinates such as poly(1,4-butylene-co-succinate), polyalkylene oxalates, polydioxanone, alkyd resins, and polycarbonates (e.g., poly(trimethylene carbonate)); polyorthoesters; polyanhydrides; polyamides, such as nylon 6, nylon 66 and/or polycaprolactam; polyimides; polysaccharides (e.g., starch, cellulose, cellulose nitrates, cellulose ethers such as methylcellulose, ethylcellulose, hydroxypropylcellulose, hydroxypropylmethylcellulose and/or carboxymethylcellulose, cellulose esters such as cellulose acetate, cellulose propionate and/or cellulose butyrate, chitosan, dextrin, maltodextrin, agar, alginic acid and/or hyaluronic acid); polypeptides (e.g., collagen, fibrin and/or fibrinogen); polyethers, such as polyethylene glycol and polypropylene glycol; polyoxymethylenes; epoxy resins; silicones; polyolefins, such as polyethylene, polypropylene, polyisobutylene and/or ethylene-alpha-olefin copolymers; fluorinated polyolefins; and blends and copolymers (including, e.g., block copolymers and/or random copolymers) thereof.

Examples of copolymers include, without limitation, ethylene vinyl acetate copolymer, ethylene vinyl alcohol copolymer, poly(lactide-co-glycolide), poly(hydroxybutyrate-co-valerate), acrylonitrile-styrene and acrylonitrile-butadiene-styrene copolymers, ethylene methyl methacrylate copolymers, poly(ethylene oxide-co-lactic acid), polyethylene-maleic anhydride copolymers, and/or poloxamers.

According to a particular embodiment, the polymer is a biocompatible polymer.

The phrase “biocompatible polymer” refers to any polymer (synthetic or natural) which when in contact with cells, tissues or body fluid of an organism does not induce adverse effects such as immunological reactions and/or rejections, cellular death, and the like. A biocompatible polymer can also be a biodegradable polymer.

Particular examples of polymers that can be used to fabricate the nanoparticles include, but are not limited to poly(lactic acid), poly(ethylene glycol) (PEG), poly(glycolic acid) (PGA) and poly lactic acid-co-glycolic acid (PLGA).

The nanoparticle may comprise more than one polymer. Typically, the nanoparticle comprises PLA. The molecular weight of the PLA used is generally in the range of about 2,000 g/mol to 300,000 g/mol. Thus, in an embodiment, the PLA used is in the range of about 1,000 g/mol to 10,000 g/mol. The average molecular weight of PLA may also be about 1,600-2,400 g/mol.

The molecular weight of the PEG used is generally in the range of 100-50,000 g/mol and more specifically between 3500-10,000 g/mol. Thus, in one embodiment, when the PEG is comprised in a PEG-PLGA copolymer, the molecule weight of the PEG is about 3000. When the PEG is comprised in TPGS, the molecular weight of PEG is about 1000.

In one embodiment, the nanoparticle comprises at least one co-polymer, examples of which include PEG-PLGA, PEG-PLA, PLA-PEG-PLA, PEG-PLA-PEG, PEG-PCL, PEG-PCL-PEG, Poly lactide-co-caprolactone (PLA-PCL), Poly(Ethylene Glycol)-Poly(l-Glutamic Acid) (PEG-PGA), methoxy-poly(ethylene glycol)-b-poly(l-lactide-co-glycolide) (mPEG-PLGA), mPEG-PCL, mPEG-PLA, methoxy-PEG-PGA, 1,2-distearoyl-sn-glycero-3-phosphoethanolamine-N-[amino(polyethylene glycol)-2000] (DSPE-PEG).

Particular combinations of polymers include PLGA and PLA (for example at a ratio of 1:4); and PLGA and PEG-PLGA (for example at a ratio of 7:1 and 4:1).

As mentioned, the outer surface of the nanoparticles of this aspect of the present invention is decorated with antigen presenting cell moieties (e.g. dendritic cell targeting moieties).

Antigen presenting cells (APC) are cells which present peptide fragments of protein antigens in association with HLA (MHC) molecules on their cell surface. Some APCs may activate antigen specific T cells.

Preferably, the APC can also stimulate CD4+ helper T cells as well as cytotoxic T cells.

Examples of APCs include, but are not limited to dendritic cells, macrophages, and B cells.

According to a particular embodiment, the APCs are dendritic cells or B cells. Most preferable are dendritic cells.

The term “dendritic cell” or “DC” refers to any member of a diverse population of morphologically similar cell types found in lymphoid or non-lymphoid tissues. These cells are characterized by their distinctive morphology and high levels of surface MHC-class II expression. DCs can be isolated from a number of tissue sources. DCs have a high capacity for sensitizing MHC-restricted T cells, and are the only antigen-presenting cells (APCs) that can activate naive T-cells. The antigens may be self-antigens that are expressed during T cell development and tolerance, and foreign antigens that are present during normal immune processes.

As used herein, an “activated DC” is a DC that has been pulsed with an antigen and is capable of activating an immune cell. The term “mature DC,” as used herein, is defined as a dendritic cell that expresses high levels of MHC class II, CD80 (B7.1) and CD86 (B7.2) molecules. In contrast, immature dendritic cells express low levels of MHC class II, CD80 (B7.1) and CD86 (B7.2) molecules but have a great capacity to take up an antigen.

The DC targeting moieties ensure that the nanoparticles are preferentially endocytosed by dendritic cells as compared to macrophages.

The DC targeting moiety may be an antibody (or fragment thereof), a protein or a peptide that binds to one or more dendritic cell surface marker(s). Such markers include, but are not limited to, DEC205, DC-SIGN, CD11c, DCIR2, Dectin-1/2, CD80/86, F4/80-like receptor, CIRE, mannose receptor, and CD36.

According to a particular embodiment, DC targeting moieties include but are not limited to carbohydrate-recognition domain ligands (such as mannose, PEG-mannose, galectin-3, tri-mannose, mannose-mimicking ligands (e.g. shikomyl), galectin-3; and Dectin-1 agonists (e.g. laminarin, PEG-laminarin, β-glucan peptides), Dectin-2 agonists; agonists of C-type lectin receptors (e.g. Langerin agonist, DC-SIGN agonists, DEC-205 agonists); CD40 agonists).

The DC targeting moieties may be linked to a polymer (preferably a hydrophilic polymer such as PLGA or PEG-PLGA). Thus, for example the present inventors contemplate using mannose-PLGA or mannose-PEG-PLGA in their nanoparticles. Methods of linking DC targeting moieties to the polymer are known in the art and include the reaction of the carboxylic acid terminal groups of PLGA with mannosamine through carbodiimide (PLGA-mannose), or the reaction of amine-PEG-mannosamine with the PLGA-NHS (mannose-PEG-PLGA).

As mentioned, the polymeric nanoparticles comprise (e.g. encapsulate) SARS-CoV-2 associated antigens.

Such antigens are typically short peptides (e.g. between 9-50 amino acids) corresponding to one or more antigenic determinants of a protein in the SARS-CoV-2 virus. The disease-associated antigen typically binds to a class I or II MHC receptor thus forming a ternary complex that can be recognized by a T-cell bearing a matching T-cell receptor binding to the MHC/peptide complex with appropriate affinity. Peptides binding to MHC class I molecules are typically about 8-14 amino acids in length. T-cell epitopes that bind to MHC class II molecules are typically about 12-30 amino acids in length. In the case of peptides that bind to MHC class II molecules, the same peptide and corresponding T cell epitope may share a common core segment, but differ in the overall length due to flanking sequences of differing lengths upstream of the amino-terminus of the core sequence and downstream of its carboxy terminus, respectively. A T-cell epitope may be classified as an antigen if it elicits an immune response. Thus, the SARS-CoV-2 antigen may be a B cell epitope, an MHC class I T cell epitope or an MHC class IIT cell epitope.

The amount of the at least one antigen used in the compositions and/or methods of the present invention depends on the antigen that is used and thus varies with each different formulation. However, the antigen should at least induce an immunoprotective response without adverse side effects. Generally the particle will contain between 0.1 to 1,000 μg of each antigen. In another aspect the particle will contain 0.1 to 500 μg of each antigen. In yet another aspect the particle will contain between 0.1 to 100 μg of each antigen. 0.1 to 50 μg of each antigen can also be used in the particle in yet another aspect.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. In one embodiment, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000;55(3):227-50.

In one embodiment, the peptides are synthesized by recombinant means.

In one embodiment, a polynucleotide agent encoding the peptide (e.g. mRNA) is present in the nanoparticle.

Preferably, at least 60%, 70%, 80%, 90%, 95%, 96%, 97%, 98%, 99% of the disease-associated antigens are encapsulated within the nanoparticle.

The polymeric nanoparticles typically comprise at least two SARS-CoV-2 derived antigens, wherein a first SARS-CoV-2 derived antigen is a MHC class I T cell epitope and a second SARS-CoV-2 derived antigen is a MHC class II T cell epitope (e.g. a B cell epitope).

In one embodiment, the MHC-II and MHC-I ligands share a common amino acid sequence. The present inventors conceive that this might represent a useful approach to generate antigen presentation processes that will lead to the propagation of synergistic antibody- and cellular-mediated immune responses able to control viral infection.

Coronavirus antigens, include the full-length protein, fragments of the protein or peptide sequences from non-structural (RNA-dependent RNA polymerases (RdRP), papain-like protease (PLpro), coronavirus main protease (3CLpro) and open reading frame (ORF1a, ORF1b, ORF3a, ORF6, ORF7a, ORF7b, ORF8, ORF10)) and structural proteins, such as spike glycoproteins (S), receptor-binding domain (RBD), membrane glycoproteins (M), as well as envelope (E) and nucleocapsid (N) proteins. Other sequences may be retrieved from SARS-CoV-2 binding domains to the cell surface receptor angiotensin-converting enzyme 2 (ACE2), L-SIGN (CD209L), transmembrane protease serine 2 (TMPRSS2) or others.

According to a specific embodiment, the SARS-CoV-2 derived antigen comprises a peptide having the amino acid sequence as set forth in SEQ ID NOs: 1-142. According to a specific embodiment, the SARS-CoV-2 derived MHC-I restricted antigen comprises a peptide having the amino acid sequence as set forth in SEQ ID NO: 14. According to a specific embodiment, the SARS-CoV-2 derived MHC-II restricted antigen comprises a peptide having the amino acid sequence as set forth in SEQ ID NO: 15.

As mentioned, the nanoparticles described herein further comprise an adjuvant.

The term “adjuvant” as used herein refers to a substance that increases the ability of an antigen to stimulate the immune system.

According to a particular embodiment, the adjuvant comprises a Toll-like receptor ligand. Preferably, the TLR ligand is encapsulated within the nanoparticle.

The Toll-like receptor (TLR) ligand may be a ligand of TLR2, a ligand of TLR3, a ligand of TLR4, a ligand of TLR5, a ligand of TLR7/8 and/or a ligand of TLR9.

Examples of TLR ligands include, but are not limited to zymosan, Polyinosinic-polycytidylic acid (Poly(I:C)), Monophosphoryl Lipid A (MPLA)), flagellin, Gardiquimod, Imiquimod (R837) Resiquimod, Inducible T-cell co-stimulator ligand (ICOSL) and CpG oligodeoxynucleotides (CpG ODN).

As used herein “CpG oligodeoxynucleotides” are short DNA sequences bearing unmethylated CpG motifs that bind to the Toll-like receptor 9 (TLR9). TLR is a receptor expressed on B cells and plasmacytoid dendritic cells causing the up regulation of MHC and other stimulatory molecules, which in turn results in more potent APC mediated T cell stimulation. Examples of CpG oligodeoxynucleotides include ODN 2006, ODN D35, ODN 1018 ISS, ODN 1758, ODN 1826 (SEQ ID NO: 3), ODN 2216, ODN 2007, ODN 1668, ODN 1720, ODN 2006, ODN 2041, OSN 7909, CpG-28 and the like.

According to a particular embodiment, the nanoparticle comprises (e.g. encapsulates both CpG ODN 1826 and Poly(I:C).

According to another embodiment, the nanoparticle comprises (e.g. encapsulates both CpG ODN 1826 and Monophosphoryl Lipid A (MPLA).

Additional adjuvants can be comprised in the nanoparticles disclosed herein. These include, but are not limited to hyaluronic acid (HA), poloxamer 407 (Pluronic F127 (PL)), 2′,3′-cGAMP, chitosan, poly-glutamic acid, poly-arginine, Dectin-1 agonist laminarin and β-glucan.

According to a particular embodiment, as well as encapsulating at least one Toll-like-receptor ligand, the nanoparticle comprises poloxamer 407 (Pluronic F127) or hyaluronic acid (HA).

According to a particular embodiment, as well as encapsulating at least one Toll-like receptor ligand, the nanoparticle comprises HA and PL.

It will be appreciated that the nanoparticles of the present invention may further comprise a surfactant.

Examples of such surface active agents include but are not limited to poly(vinyl alcohol) (PVA) and d-α-Tocopheryl polyethylene glycol 1000 succinate (TPGS).

According to a particular embodiment, the surfactant is TPGS.

As well as the components described herein above, the nanoparticles of the present invention may also encapsulate polynucleotide agents capable of downregulating the amount of a polypeptide.

Exemplary polypeptides that may be down-regulated include both soluble and non-soluble immune suppressive factors, but are not limited to TGF-β, VEGFA, PD-L1/PD-1, VEGFR1, VEGFR2, VEGFR3, IDO, RANKL, IL-10 and PGE2 receptor and pro-inflammatory cytokines and chemokines such as CXCL12, CCL2 or CCL7.

According to a particular embodiment, the polypeptide is PD-L1.

According to a particular embodiment, the polypeptide is TGF-β1.

Non-limiting examples of agents capable of down regulating expression are described in detail hereinbelow.

Down-regulation at the nucleic acid level is typically affected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding the relevant protein.

According to specific embodiments, the downregulating agent directly interacts with the relevant protein.

According to specific embodiments, the agent directly binds the relevant protein.

According to specific embodiments, the agent indirectly binds the relevant protein (e.g. binds an effector of the relevant protein).

According to specific embodiments the downregulating agent is an RNA silencing agent or a genome editing agent.

The nucleic acid agent may for complexed to a polymer in order to enhance stabilization thereof.

Exemplary polymers which can be used to complex to the nucleic acid agents (e.g. siRNA agents) include glutamate chitosan, poly-arginine, alkylated poly(α)glutamate amine (APA) and poly-(α)glutamic acid (PGA).

In one embodiment, downregulation of the relevant protein can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g. the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Exemplary RNA silencing agents include, but are not limited to dsRNA, siRNA. shRNA, miRNA, miRNA mimic, antisense agent, DNAzyme, ribozyme and RNA-guided endonuclease technology e.g. components of the CRISPR system including gRNAs.

A suitable siRNA directed against PD-L1 is set forth in SEQ ID NO: 148 (Sequence 5′-CCC ACA UAA AAA ACA GUU Gtt-3′).

It will be appreciated that, and as mentioned hereinabove, the RNA silencing agent of some embodiments of the invention need not be limited to those molecules containing only RNA, but further encompasses chemically-modified nucleotides and non-nucleotides.

The nanoparticles can be made using different methods such as attrition, pyrolysis, using thermal plasma methods, gas-phase techniques, multiple emulsion-solvent evaporation methods, gas-flow focusing, electrospray, fluidic nanoprecipitation methods, emulsion diffusion-evaporation methods, modified phase inversion/solvent diffusion methods, or sol-gel methods. These methods are described in the literature and known to those skilled in the art.

According to one embodiment, the nanoparticles are generated by a double emulsion-solvent evaporation (w/o/w) method.

Thus, in one embodiment, the polymer used to fabricate the nanoparticle (e.g. PLGA/PLA) is dissolved in a solvent (e.g. dichloromethane (DCM)). It will be appreciated that the dendritic cell targeting moiety may be conjugated to the PLGA polymer. PEG-grafted PLGA or PEG-grafted PLA may also be used to improve the hydrophilicity of the nanoparticle and to promote DC targeted delivery. Lipid adjuvants (such as MPLA, α-galactosylceramide) may also be added at this stage. The disease antigens and Toll-like receptor (TLR) ligands are added to PVA and the two mixtures combined. An emulsification step then takes place to obtain an oil in water emulsion (o/w) which is subsequently added to a surfactant (such as TPGS). A second emulsification step then takes place to form the double emulsion water-in-oil-in-water (w/o/w). Finally, the w/o/w is added to PVA or alternatively PL.

In another embodiment, the particles are generated using a continuous microfluidic assembly (e.g. NanoAssemblr® from Precision Nanosystems).

In another embodiment, lipids such as phospholipids (e.g. 1-palmitoyl-2-oleoyl-sn-glycero-3-phosphocholine (POPC) and 1,2-Dimyristoyl-sn-glycero-3-phospho-rac-(1-glycerol) sodium salt (DMPG)) or PEG-grafted phospholipids (1,2-distearoyl-sn-glycero-3-phosphoethanolamine-N-[amino(polyethylene glycol)-2000 (DSPE-PEG)) are added to the polymer solution.

In one embodiment, the nanoparticles are used as a vaccine.

As used herein, the term “vaccine” refers to a pharmaceutical preparation (pharmaceutical composition) or product that upon administration induces an immune response, in particular a cellular immune response, which recognizes and attacks the virus.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

Since the nanoparticles described herein include the use of materials that enable the stable incorporation of adjuvants and antigens, the present inventors conceive that the vaccine can be stored lyophilized (e.g. as a pre-filled syringe), without refrigeration, and for nasal administration. The lyophilized product was shown to be readily reconstituted into a suspension, which quality attributes (e.g. mean particle diameter and size distribution, surface charge, dispersibility) did not change when compared to those presented prior to the lyophilization process.

In one embodiment, the nanoparticles are used as droplets applied on the nasal mucosa upon reconstitution of the lyophilized product, to overcome mucosal barriers and deliver antigens and immune modulators at the vicinity of antigen presenting cells. In a particular embodiment, the final dosage form can be a liquid suspension readily reconstituted in saline, suitable for different administration routes, including to be applied into the nostrils as droplets.

According to a particular embodiment, the nanoparticles described herein are lyophilized.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to ensure that levels of the active ingredient are sufficient to induce or suppress the biological effect (minimal effective concentration, MEC). The MEC will vary for each preparation but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Several mouse models previously developed to address the development of therapeutic and prophylactic solutions against SARS-CoV infection can now be explored to advance drug discovery and development against COVID-19. Examples include the ACE2, TMPRSS2 and STAT1 knockout mouse models. These experimental mouse models are particularly suitable for the study of SARS-CoV-2 pathogenesis and therefore to support antivirals development, as ACE2^{29,30} and TMPRSS2^{30,31} have been identified as being related with SARS-CoV-2 entry into cells, while STAT1^{32,33} favors progressive lung disease by increasing viral replication in the lungs. BALB/c and C57BL/6 mice may be used to characterize the immune response against potential vaccine candidates, even if in the absence of disease³⁴. Particularly relevant for characterizing the human immune response triggered by SARS-CoV-2 infection and therefore to guide the design of effective and safe vaccines may be the transgenic human leucocyte antigen (HLA) class I and class II mouse models³⁵ harboring HLA genes covering high percentages of human population. Additional tools to support preclinical development include the humanized ACE2 transgenic mouse model for SARS-CoV-2 infection³⁶, such as the one developed by the Jackson Laboratories following a model previously established by Pearlman research group^{37,38}. Moreover, the mice that support the development of immune cells following the engraftment of human peripheral blood mononuclear cells (PBMC)^{39,40} or human hematopoietic stem cells (HSC)^{41,42} are also useful as an effort to translate present findings into the human settings.

A challenge study may be performed on SARS-CoV-2 mouse models (inbreed and ACE2, TMPRSS2 and STAT1 knockout mouse models) by inoculating this virus into the nostrils of vaccinated animals. In these studies, the serum, as well as nasal and lung washes are collected for serologic detection of different classes of immunoglobulins and their reactivity is confirmed against SARS-CoV-2 antigens entrapped within each vaccine candidate.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

The nanoparticles described herein protect its cargo, enabling the concomitant delivery of multiple bioactive agents to dendritic cells. Therefore, it is suitable for subcutaneous, intradermal, intramuscular, intravascular, and mucosal administrations, such as nasal, ocular, vaginal, sublingual and oral.

The nanoparticles disclosed herein are capable of being used in combination with other therapeutics. Alternatively, the additional therapeutic may be formulated (e.g. entrapped) inside the nanoparticle.

Examples of therapeutics that can be used in conjunction with the nanoparticles disclosed herein (or may be formulated inside the nanoparticle) include, but are not limited to anti-inflammatory drugs, anti-viral drug and inhibitors (e.g. viral polymerase and protease inhibitors), other modulators of immune factors, such as IL-6, IL-1, and complement protein 5 (C5), cathepsin B and cathepsin L inhibitors, combined with antibiotics.

According to a particular embodiment, the nanoparticles of the present invention are administered together with (or formulated to include within) at least one of the above disclosed agents.

The vaccines can also be potentially used to improve the activation and maturation of dendritic cells (DC) ex vivo (Plasmocytoid DC, Myeloid DC1, Myeloid DC2, monocyte-derived DC), which may be used subsequently as cell therapy. Accordingly, (as described in Conniot, J., et al. Nat Nanotechnol 14, 891-901 (2019) and in PCT Patent Application No.: PCT/IL2019/051420), Dendritic cells may be incubated with the nanoparticles described herein. The functional state of these cells may be assessed through the quantification of activation and maturation markers (CD40, CD80, CD83, CD86, and cytokines IL-12p70, IL-10, and IFN-γ). The SARS-CoV-2 peptide antigens entrapped within the nanoparticles are also suitable tools to re-activate lymphocytes, such as B and T cells contained in peripheral blood mononuclear cells (PBMC) collected from patients following apheresis. B cell activation may be determined by markers such as CD19, CD138, CD38, IgG, IgM. T cell activation may be determined by markers such as CD107, CD69, CD154, CD3, CD4, CD8, Th17. Tfh (follicular T helper cells) isolated from spleen or lymph node may be determined by CD4, CXCR5. Follicular T regulatory cells isolated from spleen and lymph nodes may be determined by analyzing FOXP3 and CXCR5 levels.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual “Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, sec, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Material and reagents. PLA (2,000 Da) with a weight-averaged molecular mass (Mw) of 2,000 was purchased from PolySciences, Inc. PLGA Resomer® RG 503H with a Mw range 24,000-38,000, poly(vinyl alcohol) (PVA, Mw 13,000-23,000 Da), dichloromethane (DCM), (deuterated) dimethyl sulfoxide (DMSO or dDMSO), dimethylformamide (DMF), 4-dimethylaminopyridine (DMAP), D-mannosamine hydrochloride, fluorescamine, paraformaldehyde (PFA) 4% (v/v), TPGS, Corning™ High binding 96 and 384 well plates, tetramethylbenzidine (TMB) ultra-sensitive, blue, horseradish peroxidase substrate D-(+)-Trehalose dihydrate, and bovine serum albumin (BSA) were purchased from Sigma-Aldrich. N-butyl poly-L-arginine hydrochloride (pARG, Mw range 3,000-3,400) was purchased from Polypeptide Therapeutic Solutions. Phosphate buffered saline (PBS, pH 7.4), Quant-iT™ RNA Assay Kit (broad range), Quant-iT™ OliGreen™ ssDNA assay kit, HEPES buffer (1 M), β-mercaptoethanol (50 mM), LIVE/DEAD™ fixable yellow dead cell stain kit (for 405 nm excitation), ACK lysing buffer and CD28 Monoclonal Antibody (37.51), eBioscience™ were purchased from Thermo Fisher Scientific. SARS-CoV-2 antigens were purchased from GeneCust, ProteoGenix SAS or Sigma-Aldrich. CpG-ODN 1826 (TCCATGACGTTCCTGACGTT (SEQ ID NO: 143)) and small interfering RNA (siRNA) anti-PD-L1 was purchased to Merck. Poly(I:C) (High Mw) VacciGrade™ was purchased from InvivoGen. Fluorochrome-labeled antibodies, permeabilization 10× and intracellular fixation buffer were purchased from BioLegend and Thermo Fisher. Elispot kit was purchased from R&D Systems Inc. Peroxidase AffiniPure Goat Anti-Mouse IgG and IgM from Jackson Immuno Research Laboratories. RBD protein was produced as reported⁶⁶. RBD variants were purchased from ProteoGenix SAS. Human ACE2 was purchased from InvivoGen.

Synthesis of NP. NP was formulated by the double emulsion-solvent evaporation method, following methods already established³⁵. A man-PLGA/PLA (2:8) blend was dissolved in DCM at 50 mg/ml. A 10% (m/v) PVA aqueous solution (100 μl) containing CpG at 0.5 mg/ml, Poly (I:C) at 1.0 mg/ml and SARS-COV2 antigens (Table 1) at 10 mg/ml was added to DCM. A 10% (m/v) PVA aqueous solution was added for empty NP. The mixture was emulsified with a microprobe ultrasonic processor for 15 sec at 20% amplitude. A 2.5% (m/v) TPGS aqueous solution (400 μl) was added, and the second emulsion was formed using the same conditions. The double emulsion was added dropwise into a 0.125% (m/v) PF-127 aqueous solution and stirred for 1 h at room temperature. Particle suspension was collected by centrifugation at 20,000 g for 45 min, 4° C. (Beckman J2-21M/E High Speed Centrifuge). Particles were washed with ultrapure water, collected by centrifugation, and finally resuspended in PBS or ultrapure water. Similarly, siRNA NP was formulated by the double emulsion-solvent evaporation method, following the above method with a prior step of N-butyl-poly-L-arginine cationic polymer complexed with siRNA. In short, 12.5 μg of siRNA was complexed with N-butyl-poly-L-arginine cationic polymer, in RNase-free ultra-pure water. Next the complex was encapsulated with MHC-I and MHC-II peptide and adjuvants as described above to reach a total of 25 μg/mouse.

Size distribution and ζ potential measurements. Particle size and polydispersity index (PdI) were determined by dynamic light scattering using the Zetasizer Nano ZS equipment (Malvern Instruments). The particles ζ potential was measured by laser Doppler velocimetry in combination with phase analysis light scattering with the same equipment. Particles were diluted in ultrapure water and the electrophoretic mobility was determined at 25° C. with the Helmholtz-Smoluchowski model by cumulative analysis. In addition, the size, PD-index and ζ potential of the lyophilized NP were measured by dynamic light scattering (DLS) (Wyatt technology). In short. NP were re-suspend in 5% D-(+)-Trehalose dihydrate (w/v in UPW) and lyophilized.

Particle morphology by Atomic Force Microscopy (AFM). Particles were diluted at 10 mg·ml⁻¹ in ultrapure water. A drop of the sample was placed onto freshly cleaved mica for 20 min and dried with pure nitrogen. Samples were analyzed in tapping mode in air at room temperature using a Nanoscope IIIa Multimode (Digital Instruments/Veeco) atomic force microscope and etched silicon tips (ca. 300 kHz) at a scan rate of ca. 1.6 Hz.

Scanning electron microscopy. Particles were diluted in trehalose 5% (m/v) and fast frozen at −80° C. f□r 2 h. Samples were dried under vacuum, first at −20° C. f□r 14 h and then at 20° C. for 2 h. Dried specimens were coated with gold on a Peltier cold-stage sputter coater and examined using a FEI Quanta 200 FEG ESEM Phillips 500 scanning electron microscope at a 5 kV accelerating voltage.

Transmission electron microscopy (TEM). Particles were diluted in PBS and placed on a carbon-coated copper grid and dried. The samples were analyzed with a Philips CM 120 Bio-Twin transmission electron microscope.

NP internalization into BMDC. To test the NP internalization in vitro, we isolated hematopoietic stem cells from bone marrow of C57BL/6J mice. C57BL/6J mice were euthanized and the bones of the hind limbs fully removed. Bone marrow cells were extracted by rinsing the bone cavity with RPMI (Thermo Fisher Scientific) medium using 25G needle. The cellular suspension was filtered by 70 μm cell strainer, red blood cell (RBC) lysis was performed (RBC lysis, biolegend). Finally, cells were suspended in RPMI supplemented with 10% (v/v) FBS, 1% (v/v) PEST, 1% (v/v) HEPES, 1% (v/v) sodium pyruvate, 0.1% (v/v) 2-Mercaptoethanol and 20 ng/ml of GM-CSF Recombinant Mouse Protein. 10⁷/10 ml cells were plated in a low attachment T flask (Sigma-Aldrich) for 7 days. After, clusters of BMDC were lightly bound to a monolayer of tightly adherent fibroblasts. BMDC were harvested and image stream flow cytometry and flow cytometry assays were performed.

ImageStream Flow Cytometry: 10⁶ BMDC were incubated with Cy5-labled empty NP (0.5 mg/ml) for 1 h. Then, treatment was removed, cells were washed with medium and further stained with LysoTracker Green DND-26 (Life technologies, Carlsbad CA, USA) and incubated for 30 min at 37° C., and with the nuclei stain Hoechst 33342 trihydrochloride trihydrate (H3570) solution (Invitrogen, Carlsbad CA, USA). Then, cells were washed 3 times and resuspended in 50 μl of 2% FBS and 2 mM EDTA in PBS solution and analyzed using an ImageStreamX Mark II Imaging Flow Cytometer for colocalization or cellular uptake by fluorescence signal evaluation. Similarity (colocalization) threshold was set as above 1.5 (AU) fluorescence signal intensity of red and green overlapping pixels.

Flow cytometry: 10⁵ BMDC were seeded in 96-well plate and incubated with Cy5-labled empty NP (1 μM Cy5 equivalent) for 0, 0.5, 1, 3, 18, 24 h. Cells were washed and resuspended in FACS buffer (0.5% BSA and 2 mM EDTA in PBS) and analyzed using Attune flow cytometer (Life Technologies) to measure the Cy5 intensity in BMDC. The results were analyzed by Kaluza 2.1 software (Beckman Coulter, USA).

EE and LC of antigens and immune potentiators. Entrapped SARS-CoV-2 antigens and adjuvants were indirectly quantified using the supernatants collected from the centrifugations. The EE (%) (equation (1)) and LC (μg/mg) (equation (2)) of SARS-COV2 antigens was determined using fluorescamine. The relative fluorescence units were measured with a Varioskan Lux Reader (Thermo Fisher) at 382/480 nm for the excitation/emission wavelengths. The amount of Poly(I:C) was determined using the Quant-iT™ RNA Assay Kit (broad range), while CpG was determined by the Quant-iT™ OliGreen™ ssDNA Assay Kit, following manufacturer's instructions. Relative fluorescence units were measured with a Varioskan Lux Reader (Thermo Fisher) at 485/520 excitation/emission wavelengths for binding of OliGreen™ reagents to CpG and at 644/673 nm excitation/emission wavelengths for RNA Assay kit.

$$\begin{matrix}EE ( \% ) = \frac{\begin{matrix}initial amount of biomolecule - amount of \\ biomolecule in the supernatant\end{matrix}}{initial amount of biomolecule} \times 100 & ( 1 ) \\ LC ( μg {mg}^{- 1} ) = \frac{\begin{matrix}initial amount of biomolecule - amount of \\ biomolecule in the supernatant\end{matrix}}{total amount of polymer} & ( 2 )\end{matrix}$$

Cell viability assay. BMDC were obtained as previously described, and were seeded in 96 well plate, 10⁵ cells/well. The cells were treated with increasing concentrations of Empty NP (125, 250, 500, 1000 μg/ml) and their viability was tested by cell proliferation kit II (XTT) (Sigma-Aldrich, cat #11465015001) in several time points (3, 6, 20, 44 h). At the end-point cells were incubated with XTT reagent, according to the manufacturer instructions, for 4 h at 37° C. and samples O.D. was measured by a SpectraMax plate reader (Molecular Devices) at 450 nm.

Animal studies. All the animal procedures were performed in compliance with the Portuguese competent authority for animal protection (Direcção Geral de Alimentação e Veterinária) and Sackler Faculty of Medicine, Tel Aviv University guidelines. The protocols (0421/000/000/2021 and 01-20-060) were approved by the Institutional Animal Care and Use Committees at the University of Lisbon or at the Tel Aviv University and performed in accordance with National Institutes of Health guidelines. Male C57BL/6J mice (8-weeks-old) were purchased from Instituto Gulbenkian de Ciência (IGC) or Envigo Ltd and housed in the animal facility of the Faculty of Pharmacy, University of Lisbon or at Tel Aviv University. Mouse body weight change was monitored three times per week until day 28 after 1^{st} immunization and two times per week after. Mice were euthanized according to ethical protocols.

In vivo safety. All behavioral studies, open field and RotaRod, conducted in the Myers Neuro-Behavioral Core Facility (Tel-Aviv University, Israel). C57BL/6J male mice (8-week-old) were randomized into eight groups (N=10). The treatment (NV-7, a total of 400 μg peptide/mouse) was injected to the mice every 7 days to a total of two injections. To detect the behavioral change, mice were tested before and after each treatment.

Open-field: The open field consisted of 50×50×40 cm plexiglass arena with a white floor and light intensity of 300 lx. Each mouse was placed in a corner of the arena and allowed free exploration of the arena for 15 min. Mouse behavior was continuously recorded by a video camera placed over the structure and analyzed using EthoVision-XT software (Noldus Information Technology).

RotaRod: Mice were placed on a 5-lane accelerating Rotarod (Ugo Basile, Italy) for balance assessment. Each mouse was placed on a 3-cm-diameter horizontal rod elevated 16 cm from the ground. Mice were subjected to five trials in every session, of which the three highest score tests trials were averaged. A trial begins with the rod spinning at 4 RPM and gradually accelerating by a factor of 0.5 cm/s every 5 s to a maximum of 50 RPM. The latency until falling from the rod was measured and analyzed. Following the RotaRod and open-field tests, one week post the second NV injection, mice were euthanized, and blood was collected to test the effect of NV treatment in vivo on mice blood chemistry and blood count. Blood samples were analyzed by AML Ltd (Herzliya, Israel).

Immunization of animals with SARS-CoV-2 antigens. For immunization studies, 8-week-old C57BL/6J male mice were randomized into treatment groups (N=8-15). Treatments (100 μl) were injected into each mouse by hock immunization via s.c. injection proximal to popliteal lymph nodes. A half dose (50 μl) was injected into the right side and the other half into the left side. Each dose contained 400 μg of antigen (200 μg of P14 and 200 μg of P15) plus 20 μg of CpG and 40 μg of Poly (I:C), either free in solution or entrapped in 2 mg of particles (20 mg/ml). To assess the ability of NV to induce mucosal immune response, an intranasal booster (50 μl) was administered to mice, 3 weeks following the subcutaneous administration of the first dose. A comparison with the commercially-available approved Pfizer-BioNTech COVID-19 Vaccine (also known as COMIRNATY) was performed. Bronchoalveolar lavage fluid (BALF) was collected one weeks after the booster nasal immunization and the levels of secretory IgA were quantified by ELISA.

ELISA. ELISA was performed for the detection of peptides in Table 1 or SARS-CoV-2 RBD-specific antibodies in immunized mouse sera. Corning High binding 96-well plates were precoated with peptides (10 μg/ml) or RBD protein (1 μg/ml) overnight at 4° C. in carbonate buffer (pH 9.6). Plates were washed three times with PBS-T (PBS+0.05% Tween-20) and blocked with 3% BSA (Sigma-Aldrich, #A8022) in PBS-T (PBS+0.1% Tween-20) for 2 h at 37° C. After three washes with PBS-T. plates were incubated with serially diluted mouse sera or BALF in PBS-T/1% BSA for 1 h at 24° C. Following washing, Peroxidase AffiniPure Goat Anti-Mouse IgG, IgA or IgM (Jackson Immuno Research Laboratories) were added for 1 h at 24° C. The plates were washed with PBS-T and reactions were developed with TMB ultra-sensitive, blue, horseradish peroxidase substrate (Sigma-Aldrich). The reaction was stopped by adding 0.5 M of sulfuric acid. Plates were read at 405 nm absorbance using the Varioskan Lux Reader (Thermo Fisher). Antibody titers were calculated as the highest serum dilution with an optical density (OD) value above 2 times the average OD of the negative control.

Cellular Immune Responses Assessed by flow cytometry. On day 28, mice were euthanized, the spleens harvested and the splenocytes isolated. Splenocytes from each group were seeded at 3-4×10⁶ cells per in well at 6-well plates. Splenocytes were cultured with 100 μg·ml⁻¹ of P14 and P15 and 2 μg/ml of CD28 or medium only (negative control). After incubation at 37° C. for 6 h in the presence of Brefeldin for the last 4 h of culturing, cells were labelled for surface markers (CD3, CD4, CD8, CD25, CD107b and CD127 [Biolegend]) and the LIVE/DEAD Yellow indicator dye (Life Technologies) was added. The intracellular cytokines were detected by antibodies specific for T helper 1 (TH1) cytokines interferon gamma (IFN-γ), tumor necrosis factor alpha (TNF-α), and interleukin (IL)-2; T helper 2 (T_{H}2) cytokines IL-4, IL6 and IL-10 (Biolegend). The samples were processed using the Cytek Aurora flow cytometer (Cytek). Data were analyzed using FlowJo software version Xv10 (Tree Star Inc).

Functional assessment of T cells. For the ELISpot assay, on day 0 mice were randomized into 4 groups, and treated according to the schedule used in FIG. 5. On day 28, mice were euthanized, the spleens harvested and the splenocytes isolated. Splenocytes were seeded at 2×10⁵ cells per well in 96-well plates coated with IFN-γ antibody (R&D Systems Inc.) and incubated for 20 h with 2 μg/ml of CD28 (Invitrogen) and 1 mg/ml of peptides 14 and 15. The secreted and captured IFN-γ was subsequently detected using a biotinylated antibody specific for IFN-γ and an alkaline-phosphatase conjugated to streptavidin. After the addition of the substrate solution, a blue precipitate formed and appeared as spots at the sites of cytokine localization. Automated spot quantification was performed using the Cytation 7 (Biotek).

Challenge of immunized animals. 8-week-old C57BL/6J male mice were randomized into 5 treatment groups; PBS, Free, Empty NP. Adjuvant NP and NV (N=8-10 per group). Then, mice were immunized with 200 μl of: PBS, Free (200 μg of P14, 200 μg of P15, 20 μg of CpG and 40 μg of Poly:IC, free in PBS), Empty NP (2 mg of particles in PBS), Adjuvant NP (20 μg of CpG and 40 μg of Poly: IC, entrapped in 2 mg of particles) and NV (200 μg of P14, 200 μg of P15, 20 μg of CpG and 40 μg of Poly:IC, entrapped in 2 mg of particles). Two immunizations were administrated via s.c. injection proximal to popliteal lymph nodes on days 0 and 21. On day 95, all mice groups were challenged with an i.v. injection of 200 μg of P14, 200 μg of P15, 20 μg of CpG and 40 μg of Poly:IC, free in PBS. Blood was collected from mice check on days 0, 7, 14, 21, 28, 35, 49, 63, 77, 91 and 101 for antibody detection by ELISA. On day 101, mice were euthanized, the spleens harvested and the splenocytes isolated. Splenocytes from each group were labelled for surface markers (CD8, CD3, CD4, CD69, CD44, CD62L B220, CD38, and IgG [miltenyi]) and the Zombie LIVE/DEAD indicator dye (Invitrogen) was added. The samples were processed using the Cytek Aurora flow cytometer (Cytek). Data were analyzed using SpectroFlo.Ink software.

### Quantitative Real Time RT-PCR

Frozen spleen samples were homogenized using a motor-driven grinder on TRIzol™ reagent (Invitrogen, Thermo Fisher Scientific, Waltham, MA, USA), and then total RNA was extracted following to the manufacturer's instructions. Total RNA was quantified in a Qubit™ 2.0 fluorometer (Invitrogen, Thermo Fisher Scientific) and 1.5 μg RNA were converted into cDNA using NZY First-Strand cDNA Synthesis Kit (NZYTech, Lisbon, Portugal), according to the manufacturer's protocol. Quantitative real-time RT-PCR (qPCR) was performed in the QuantStudio™ 7 Flex Real-Time PCR System (Applied Biosystems™, Thermo Fisher Scientific). qPCR was performed in 5 μL duplicate reactions on a 384-well QuantStudio 7 Flex Real-Time PCR System (Applied Biosystems, Thermo Fisher Scientific), using the 2×SensiFAST SYBR Hi-ROX kit (Bioline, Meridian Bioscience, Inc., Cincinnati, OH, USA), following manufacturer's protocol. The following primer sequences were used: for Pd-11 gene 5′ ATT CTC TGG TTG ATT TTG CGG TA 3 (forward—SEQ ID NO: 144) and 5′ TTC AGA TCA CAG ACG TCA AGC TG 3′ (reverse—SEQ ID NO: 145); for the hypoxanthine phosphoribosyltransferase (Hprt) gene, 5′ GGT GAA AAG GAC CTC TCG AAG TG 3′ (SEQ ID NO: 146; forward) and 5′ ATA GTC AAG GGC ATA TCC AAC AAC A 3′ (SEQ ID NO: 147, reverse). The relative amount of Pd-11 was calculated based on the standard curve and was normalized to the level of Hprt, being expressed as fold change from PBS controls.

sVNT assay. A Corning High binding 96-well plate was precoated with WT RBD protein or RBD variants (1 μg/ml) overnight at 4° C. in carbonate buffer (pH 9.6). Plates were washed three times with PBS-T (PBS+0.05% Tween-20) and blocked with 3% BSA (Sigma-Aldrich, #A8022) in PBS-T (PBS+0.1% Tween-20) for 2 h at 37° C. After three washes with PBS-T, plates were incubated with ACE2-biotin (InvivoGen) and mouse sera (1:2) (final volume of 25 μl) in PBS-T/1% BSA for 1 h at 24° C. Following washing, streptavidin-HRP (Jackson Immuno Research Laboratories) was added for 1 h at 24° C. The plates were washed with PBS-T and reactions were developed with TMB ultra-sensitive, blue, horseradish peroxidase substrate (Sigma-Aldrich). The reaction was stopped by adding 0.5 M of sulfuric acid. The absorbance readings at 405 nm were acquired using the Varioskan Lux Reader (Thermo Fisher). The OD values were converted to a common scale 0-100. Inhibition (%) was measured through the following metrics: [1−(OD value of unknown sample/OD value of Max interaction)×100.

### In Vitro Cell Neutralization Assay

Virus-like particles (VLP) pseudotyped with the SARS-CoV-2 S protein were prepared by co-transfecting HEK293T cells with SARS-CoV-2 spike ORF expression vector pCMV3-SARS-CoV-2 Spike (Sino Biological; #VG40589-UT) together with packaging vector pCMVΔR8.2 and reporter plasmid pHR'-CMV-turboGFP-Neo (kindly provided by Prof. Ben-Baruch, Tel-Aviv University) using the calcium phosphate method. Cell supernatants (CS) containing VLP were collected 48 h post-transfection and cleared by centrifugation and filtration through a 0.45 μm membrane.

To determine the neutralization activity of the serum samples from animals, HEK293T cells stably expressing human ACE2 (hACE2) were plated in 96-well plates (Corning) at 5×10⁴ cells/well overnight. Mice sera were mixed with fresh VLP-containing CS at a ratio of 1:100 and incubated at the presence of 8 μg/ml polybrene at 37° C. for 1 h. As controls for neutralizing activity, Spike-VLP were incubated with SARS-CoV-2 spike neutralizing antibodies (Active Motif; #am001414) or isotype control (Biolegend). Then, HEK293T-hACE2 cells were infected with 100 μl/well of sera-VLP CS mixture and centrifuged at 1200×g for 1.5 h at 30° C. Following the spinoculation protocol, sera-VLP CS mixture was removed, and fresh medium was added to the cells. The cells were monitored daily for GFP signal and imaged after 5 days with an EVOS FL Auto cell imaging system (ThermoFisher Scientific, Massachusetts, USA). Several images of independent fields per well were taken and the numbers of GFP-positive cells were counted by ImageJ software.

Particular nano-vaccine formulations were generated as summarized in Table 1.

TABLE 1

| | | | SEQ | | | Epitope | |
|---|---|---|---|---|---|---|---|
| | SARS-CoV- | | ID | | | T | B |
| NV | 2 Region | Peptide Sequence | NO: | MHCI | MHCII | cell | cell |
| 1 | S (327-335) | VRFPNITNL | 1 | √ | — | √ | — |
| | S (802-819) | FSQILPDPSKPSKRSFIE | 2 | — | √ | — | √ |
| 2 | N (322-331) | MEVTPSGTWL | 3 | √ | — | √ | — |
| | N (168-182) | PKGFYAEGSRGGSQA | 4 | — | √ | √ | √ |
| 3 | S (996-1004) | LITGRLQSL | 5 | √ | — | √ | √ |
| | S (891-904) | GAALQIPFAMQMAY | 6 | — | √ | √ | √ |
| 4 | S (820-829) | DLLFNKVTLA | 7 | √ | — | √ | — |
| | S (389-403) | DLCFTNVYADSFVIR | 8 | — | √ | √ | √ |
| 5 | N (96-105) | GGDGKMKDLS | 9 | √ | — | √ | — |
| | N (156-170) | AIVLQLPQGTTLPKG | 10 | — | √ | √ | — |
| 6 | M (198-206) | RYRIGNYKL | 11 | √ | — | √ | — |
| | M (176-190) | LSYYKLGASQRVAGD | 12 | — | √ | √ | — |
| 7 | S (489-504) | FPLQSYGFQPTNGVG | 13 | — | √ | √ | — |
| | S (453-461) | YRLFRKSNL | 14 | √ | — | √ | — |
| 8 | S (453-461) | YRLFRKSNL | 14 | √ | — | √ | — |
| | S (481-500) | NGVEGENCYFPLQSYGFQPT | 15 | — | √ | √ | √ |
| 9 | S (491-500) | PLQSYGFQPT | 16 | √ | — | √ | — |
| | S (491-500) | NGVEGFNCYFPLQSYGFQPT | 15 | — | √ | √ | √ |
| 10 | S (481-490) | NGVEGFNCYF | 17 | √ | — | √ | — |
| | S (481-500) | NGVEGFNCYFPLQSYGFQPT | 15 | — | √ | √ | √ |
| 11 | S (484-493) | EGFNCYFPLQ | 18 | √ | — | √ | — |
| | S (481-500) | NGVEGFNCYFPLQSYGFQPT | 15 | — | √ | √ | √ |

NV-Nanovaccine Candidate;
MHC-Major Histocompatibility Complex;
S-Spike Protein;
N-Nucleocapsid Protein;
M-Membrane Protein.

Additional peptide sequences contemplated by the present invention include the T cell epitope SKVGGNYNY (SEQ ID NO: 19), derived from SARS-CoV-2 Spike protein, the MHC class II epitope PKGFYAEGSRGGSQASSR (SEQ ID NO: 20) derived from the nucleocapsid protein and the MHC class I epitope GAALQIPFAMQMAYRF (SEQ ID NO: 21) derived from the spike protein. Additional peptide sequences are summarized in Table 2, herein below.

TABLE 2

| | | | T cell | T cell | | |
|---|---|---|---|---|---|---|
| | | | Epitope | Epitope | | |
| | B cell Epitope | B cell Epitope | SARS- | SARS- | MHC | Identity |
| Protein | SARS-CoV | SARS-CoV-2 | CoV | CoV-2 | Allele | (%) |
| N | | | ALNTP | ALNTPK | HLA- | 100 |
| | | | KDHI | DHI | A*02:01 | |
| | | | (SEQ ID | (SEQ ID | | |
| | | | NO: 22) | NO: 22) | | |
| | | | LALLLL | LALLLL | | 100 |
| | | | DRL | DRL | | |
| | | | (SEQ ID | (SEQ ID | | |
| | | | NO: 23) | NO: 23) | | |
| | | | LLLDRL | LLLDRL | | 100 |
| | | | NQL | NQL | | |
| | | | (SEQ ID | (SEQ ID | | |
| | | | NO: 24) | NO: 24) | | |
| | | | RLNQL | RLNQLE | | 89 |
| | | | ESKV | SKM | | |
| | | | (SEQ ID | (SEQ ID | | |
| | | | NO: 25) | NO: 26) | | |
| | | | GETALA | GDAAL | HLA- | 80 |
| | | | LLLL | ALLLL | B*40:01 | |
| | | | (SEQ ID | (SEQ ID | | |
| | | | NO: 27) | NO: 28) | | |
| | | | MEVTP | MEVTPS | | 100 |
| | | | SGTWL | GTWL | | |
| | | | (SEQ ID | (SEQ ID | | |
| | | | NO: 29) | NO: 29) | | |
| | KHIDAYKTFPPTEPKK | NKHIDAYKTFPPTEPK | | | | 100 |
| | DKKKKTDEAQPLPQR | KDKKKKTDEAQPLPQ | | | | |
| | QKKQPTVTLLPAADM | RQKKQPTVTLLPAAD | | | | |
| | DD (SEQ ID NO: 30) | M (SEQ ID NO: 31) | | | | |
| | NNAATVLQLPQGTTL | NNNAATVLQLPQGTT | | | | 95 |
| | PKGFYA (SEQ ID NO: | LPKGF (SEQ ID NO: | | | | |
| | 32) | 33) | | | | |
| | | | DAALA | DAALA | | 100 |
| | | | LLLLDR | LLLLDR | | |
| | | | LNQL | LNQL | | |
| | | | (SEQ ID | (SEQ ID | | |
| | | | NO: 34) | NO: 34) | | |
| | ALALLLLDRLNQLESK | ALALLLLDRLNQLES | | | | 100 |
| | V (SEQ ID NO: 35) | KV (SEQ ID NO: 35) | | | | |
| | | | MSRIG | MSRIG | | 100 |
| | | | MEVTP | MEVTPS | | |
| | | | SGTWL | GTWL | | |
| | | | (SEQ ID | (SEQ ID | | |
| | | | NO: 36) | NO: 36) | | |
| | MEVTPSGTWLTYHGA | MEVTPSGTWLTYHG | | | | 100 |
| | IK (SEQ ID NO: 37) | AIK (SEQ ID NO: 37) | | | | |
| | | | PINTNS | PINTNS | | 89 |
| | | | SPD | GPD | | |
| | | | (SEQ ID | (SEQ ID | | |
| | | | NO: 38) | NO: 39) | | |
| | | | LSPRW | LSPRW | | 100 |
| | | | YFYYL | YFYYL | | |
| | | | GTGPE | GTGPEA | | |
| | | | ASL | SL (SEQ | | |
| | | | (SEQ ID | ID NO: | | |
| | | | NO: 40) | 40) | | |
| | | | FYYLG | FYYLGT | | 100 |
| | | | TGPEAS | GPEASL | | |
| | | | LPYG | PYG | | |
| | | | (SEQ ID | (SEQ ID | | |
| | | | NO: 41) | NO: 41) | | |
| | | | SPRWY | SPRWYF | | 100 |
| | | | FYYLG | YYLGT | | |
| | | | TGPEA | GPEA | | |
| | | | (SEQ ID | (SEQ ID | | |
| | | | NO: 42) | NO: 42) | | |
| | | | YFYYL | YFYYL | | 100 |
| | | | GTGPE | GTGPEA | | |
| | | | ASL | SL (SEQ | | |
| | | | (SEQ ID | ID NO: | | |
| | | | NO: 43) | 43) | | |
| | | | QFAPSA | QFAPSA | | 100 |
| | | | SAF | SAF | | |
| | | | (SEQ ID | (SEQ ID | | |
| | | | NO: 44) | NO: 44) | | |
| | | | KHWPQ | KHWPQI | | 100 |
| | | | IAQFAP | AQFAPS | | |
| | | | SASAFF | ASAFF | | |
| | | | (SEQ ID | (SEQ ID | | |
| | | | NO: 45) | NO: 45) | | |
| | KHWPQIAQFAPSASAF | KHWPQIAQFAPSASA | KHWPQ | KHWPQI | | 100 |
| | F (SEQ ID NO: 46) | FF (SEQ ID NO: 46) | IAQFAP | AQFAPS | | |
| | | | SASAFF | ASAFF | | |
| | | | (SEQ ID | (SEQ ID | | |
| | | | NO: 46) | NO: 46) | | |
| | | | AQFAPS | AQFAPS | | 100 |
| | | | ASAFFG | ASAFFG | | |
| | | | MSR | MSR | | |
| | | | (SEQ ID | (SEQ ID | | |
| | | | NO: 47) | NO: 47) | | |
| | | | AQFAPS | AQFAPS | | 100 |
| | | | ASAFFG | ASAFFG | | |
| | | | MSRIG | MSRIG | | |
| | | | M (SEQ | M (SEQ | | |
| | | | ID NO: | ID NO: | | |
| | | | 48) | 48) | | |
| | | | AIVLQL | AIVLQL | | 100 |
| | | | PQGTTL | PQGTTL | | |
| | | | PKG | PKG | | |
| | | | (SEQ ID | (SEQ ID | | |
| | | | NO: 49) | NO: 49) | | |
| S | FGAGAALQIPFAMQM | FGAGAALQIPFAMQM | GAALQI | GAGAA | HLA- | |
| | AYRFNGI (SEQ ID NO: | AYRFNGI (SEQ ID NO: | PFAMQ | LQIPFA | DRA*01:01 | |
| | 50) | 50) | MAYRF | MQMAY | | |
| | | | (SEQ ID | RF (SEQ | | |
| | | | NO: 51) | ID NO: | | |
| | | | | 52) | | |
| | | | FIAGLI | FIAGLIA | HLA- | 100 |
| | | | AIV | IV (SEQ | A*02:01 | |
| | | | (SEQ ID | ID NO: | | |
| | | | NO: 53) | 53) | | |
| | | | LITGRL | LITGRL | | 100 |
| | | | QSL | QSL | | |
| | | | (SEQ ID | (SEQ ID | | |
| | | | NO: 54) | NO: 54) | | |
| | | | NLNESL | NLNESL | | 100 |
| | | | IDL | IDL | | |
| | | | (SEQ ID | (SEQ ID | | |
| | | | NO: 55) | NO: 55) | | |
| | | | ALNTL | ALNTLV | | 100 |
| | | | VKQL | KQL | | |
| | | | (SEQ ID | (SEQ ID | | |
| | | | NO: 56) | NO: 56) | | |
| | | | RLNEV | RLNEV | | 100 |
| | | | AKNL | AKNL | | |
| | | | (SEQ ID | (SEQ ID | | |
| | | | NO: 57) | NO: 57) | | |
| | | | VLNDIL | VLNDIL | | 100 |
| | | | SRL | SRL | | |
| | | | (SEQ ID | (SEQ ID | | |
| | | | NO: 58) | NO: 58) | | |
| | | | KSFEID | KSFTVE | HLA- | 78 |
| | | | KGIYQT | KGIYQT | DRB1*04:01 | |
| | | | SNFRV | SNFRVQ | | |
| | | | V (SEQ | (SEQ ID | | |
| | | | ID NO: | NO: 59) | | |
| | | | 59) | | | |
| | | | QLIRAA | QLIRAA | | 100 |
| | | | EIRASA | EIRASA | | |
| | | | NLAAT | NLAAT | | |
| | | | K (SEQ | K (SEQ | | |
| | | | ID NO: | ID NO: | | |
| | | | 60) | 60) | | |
| | | | | YLQPRT | HLA- | |
| | | | | FLL | A*02:01 | |
| | | | | (SEQ ID | | |
| | | | | NO: 61) | | |
| | | | GAISSV | GAISSV | HLA- | 100 |
| | | | LNDILS | LNDILS | A*02:01 | |
| | | | RLD | RLD | | |
| | | | (SEQ ID | (SEQ ID | | |
| | | | NO: 62) | NO: 62) | | |
| | AISSVLNDILSRLDKV | AISSVLNDILSRLDKV | | | | 100 |
| | E (SEQ ID NO: 63) | E (SEQ ID NO: 63) | | | | |
| | IDRLITGRLQSLQTY | IDRLITGRLQSLQTY | | | | 100 |
| | (SEQ ID NO: 64) | (SEQ ID NO: 64) | | | | |
| | | | LITGRL | LITGRL | HLA- | 100 |
| | | | QSL | QSL | A*02:01 | |
| | | | (SEQ ID | (SEQ ID | | |
| | | | NO: 65) | NO: 65) | | |
| | RLITGRLQSLQTYVTQ | RLITGRLQSLQTYVTQ | | | | 100 |
| | Q (SEQ ID NO: 66) | Q (SEQ ID NO: 66) | | | | |
| | | | RLQSL | RLQSLQ | HLA- | 100 |
| | | | QTYV | TYV | A*02:01 | |
| | | | (SEQ ID | (SEQ ID | | |
| | | | NO: 67) | NO: 67) | | |
| | EAEVQIDRLITGRLQS | EAEVQIDRLITGRLQS | | | | 100 |
| | L (SEQ ID NO: 68) | L (SEQ ID NO: 68) | | | | |
| | PWYVWLGFIAGLIAIV | PWYVWLGFIAGLIAI | | | | 100 |
| | M (SEQ ID NO: 69) | VM (SEQ ID NO: 69) | | | | |
| | | | WLGFI | WLGFIA | | 100 |
| | | | AGLIAI | GLIAIV | | |
| | | | VMVT | MVT | | |
| | | | (SEQ ID | (SEQ ID | | |
| | | | NO: 70) | NO: 70) | | |
| | | | LGFIAG | LGFIAG | | 100 |
| | | | LIAIVM | LIAIVM | | |
| | | | VTI | VTI | | |
| | | | (SEQ ID | (SEQ ID | | |
| | | | NO: 71) | NO: 71) | | |
| | DAVDCSQNPLAELKC | DAVDCALDPLSETKC | | | | 69 |
| | SVKSFEIDKGIYQTSNF | TLKSFTVEKGIYQTSN | | | | |
| | (SEQ ID NO: 72) | (SEQ ID NO: 73) | | | | |
| | | ISGINASVVNIQKEIDR | | ISGINAS | | |
| | | LNE (SEQ ID NO: 74) | | VVNIQK | | |
| | | | | EIDRLN | | |
| | | | | E (SEQ | | |
| | | | | ID NO: | | |
| | | | | 74) | | |
| | | | | YEQYIK | HLA- | |
| | | | | WPWY | B*18:01 | |
| | | | | (SEQ ID | HLA- | |
| | | | | NO: 75) | B*40:01 | |
| | | | | | HLA- | |
| | | | | | B*44:02 | |
| | | | | | HLA- | |
| | | | | | B*44:03 | |
| | | | | | HLA- | |
| | | | | | B*45:01 | |
| S | | PSKPSKRSFIEDLLFN | | PSKPSK | | |
| | | KV (SEQ ID NO: 76) | | RSFIED | | |
| | | | | LLFNKV | | |
| | | | | (SEQ ID | | |
| | | | | NO: 76) | | |
| | EIDRLNEVAKNLNESL | EIDRLNEVAKNLNES | | | | 100 |
| | IDLQELGKYEQY (SEQ | LIDLQELGKYEQY | | | | |
| | ID NO: 77) | (SEQ ID NO: 77) | | | | |
| | | NLCPFGEVFNATRFA | | | | |
| | | SVYAW (SEQ ID NO: | | | | |
| | | 78) | | | | |
| | | ADYSVLYNSASFSTF | | | | |
| | | KCYGVSPTKLNDLCF | | | | |
| | | T (SEQ ID NO: 79) | | | | |
| | | | | VVLSFE | | |
| | | | | LLHAPA | | |
| | | | | TVC | | |
| | | | | (SEQ ID | | |
| | | | | NO: 80) | | |
| | | | | KCYGV | | |
| | | | | SPTKLN | | |
| | | | | DLCF | | |
| | | | | (SEQ ID | | |
| | | | | NO: 81) | | |
| | | | | KVGGN | | |
| | | | | YNYLY | | |
| | | | | RLFRKS | | |
| | | | | (SEQ ID | | |
| | | | | NO: 82) | | |
| | | GGNYNYLYRLFRKSN | | GGNYN | | |
| | | (SEQ ID NO: 83) | | YLYRLF | | |
| | | | | RKSN | | |
| | | | | (SEQ ID | | |
| | | | | NO: 83) | | |
| | | EVRQIAPGQTGKIAD | | EVRQIA | | |
| | | (SEQ ID NO: 84) | | PGQTG | | |
| | | | | KIAD | | |
| | | | | (SEQ ID | | |
| | | | | NO: 84) | | |
| | | | CVADY | CVADY | HLA- | 100 |
| | | | SVLY | SVLY | A*01:01 | |
| | | | (SEQ ID | (SEQ ID | HLA- | |
| | | | NO: 85) | NO: 85) | A*26:01 | |
| | | | | | HLA- | |
| | | | | | A*29:02 | |
| | | | | | HLA- | |
| | | | | | A*30:02 | |
| | | | | RLFRKS | HLA- | |
| | | | | NLK | A*03:01 | |
| | | | | (SEQ ID | HLA- | |
| | | | | NO: 86) | A*11:01 | |
| | | | | VIRGDE | | |
| | | | | VRQIAP | | |
| | | | | G (SEQ | | |
| | | | | ID NO: | | |
| | | | | 87 | | |
| | | NVYADSFVIRGDEVR | | | | |
| | | QIAPG (SEQ ID NO: | | | | |
| | | 88) | | | | |
| | | | | VIRGDE | | |
| | | | | VRQIAP | | |
| | | | | G (SEQ | | |
| | | | | ID NO: | | |
| | | | | 89) | | |
| | | CYFPLQSYGFQPTNG | | | | |
| | | VGYQPY (SEQ ID NO: | | | | |
| | | 90) | | | | |
| | | TESNKKFLPFQQFGR | | | | |
| | | DIA (SEQ ID NO: 91) | | | | |
| S | | | | NYNYL | HLA- | |
| | | | | YRLF | A*24:02 | |
| | | | | (SEQ ID | | |
| | | | | NO: 92) | | |
| M | MADNGTITVEELKQL | MADSNGTITVEELKK | | | | 92 |
| | LEQWNLVIG (SEQ ID | LLEQWNLVI (SEQ ID | | | | |
| | NO: 93) | NO: 94) | | | | |
| | PLMESELVIGAVIIRGH | PLLESELVIGAVILRG | | | | 90 |
| | LRMA (SEQ ID NO: 95) | HLRI (SEQ ID NO: 96) | | | | |
| | | | KKLLE | KQLLEQ | | 89 |
| | | | QWNL | WNL | | |
| | | | (SEQ ID | (SEQ ID | | |
| | | | NO: 97) | NO: 98) | | |
| | GFAAYNRYRIGNYKL | GFAAYNRYRIGNYKL | GFAAY | GFAAY | | 100 |
| | (SEQ ID NO: 99) | (SEQ ID NO: 99) | NRYRIG | NRYRIG | | |
| | | | NYKL | NYKL | | |
| | | | (SEQ ID | (SEQ ID | | |
| | | | NO: 99) | NO: 99) | | |
| | NRYRIGNYKLNTDHA | NRYRIGNYKLNTDHA | NRYRIG | NRYRIG | | 100 |
| | (SEQ ID NO: 100) | (SEQ ID NO: 100) | NYKLN | NYKLN | | |
| | | | TDHA | TDHA | | |
| | | | (SEQ ID | (SEQ ID | | |
| | | | NO: | NO: | | |
| | | | 100) | 100) | | |
| | DSGFAAYNRYRIGNY | DSGFAAYNRYRIGNY | | | | 100 |
| | KLNTDHAG (SEQ ID | KLNTDHAG (SEQ ID | | | | |
| | NO: 101) | NO: 101) | | | | |
| | | | LSYYK | LSYYKL | HLA- | 100 |
| | | | LGASQ | GASQR | DRB1*01:01 | |
| | | | RVGTD | VGTD | | |
| | | | (SEQ ID | (SEQ ID | | |
| | | | NO: | NO: | | |
| | | | 102) | 102) | | |
| | | | TLSYY | TLSYYK | HLA- | 100 |
| | | | KLGAS | LGASQR | DRB1*01:01 | |
| | | | QRVGT | VGT | | |
| | | | (SEQ ID | (SEQ ID | | |
| | | | NO: | NO: | | |
| | | | 103) | 103) | | |
| | TSRTLSYYKLGASQR | TSRTLSYYKLGASQR | | | | 100 |
| | V (SEQ ID NO: 104) | V (SEQ ID NO: 104) | | | | |
| | | | TLACF | TLACFV | HLA- | 100 |
| | | | VLAAV | LAAV | A*02:01 | |
| | | | (SEQ ID | (SEQ ID | | |
| | | | NO: 105) | NO: 105) | | |
| | | | GLMWL | GLMWL | HLA- | 89 |
| | | | SYFV | SYFI | A*02:01 | |
| | | | (SEQ ID | (SEQ ID | | |
| | | | NO: 106) | NO: 107) | | |
| | | | HLRMA | HLRIAG | Class I | 78 |
| | | | GHSL | HHL | | |
| | | | (SEQ ID | (SEQ ID | | |
| | | | NO: 108) | NO: 109) | | |
| E | | | MYSFV | MYSFVS | | 100 |
| | | | SEETGT | EETGTL | | |
| | | | LIVN | IVN | | |
| | | | (SEQ ID | (SEQ ID | | |
| | | | NO: 110) | NO: 110) | | |
| | | | SVLLFL | SVLLFL | | 100 |
| | | | AFVVF | AFVVFL | | |
| | | | LLVT | LVT | | |
| | | | (SEQ ID | (SEQ ID | | |
| | | | NO: 111) | NO: 111) | | |
| | | | FLLVTL | FLLVTL | | 100 |
| | | | AILTAL | AILTAL | | |
| | | | RLC | RLC | | |
| | | | (SEQ ID | (SEQ ID | | |
| | | | NO: 112) | NO: 112) | | |
| | | | ALRLC | ALRLCA | | 100 |
| | | | AYCCNI | YCCNIV | | |
| | | | VNVS | NVS | | |
| | | | (SEQ ID | (SEQ ID | | |
| | | | NO: 113) | NO: 113) | | |
| ORF3a | | | RFFTLG | RIFTIGT | HLA- | 40 |
| | | | SITAQP | VTLKQ | B*58:01 | |
| | | | VKI | GEI | | |
| | | | (SEQ ID | (SEQ ID | | |
| | | | NO: 114) | NO: 115) | | |
| | | | SITAQP | TVTLKQ | HLA- | 22 |
| | | | VKI | GEI | B*58:01 | |
| | | | (SEQ ID | (SEQ ID | | |
| | | | NO: 116) | NO: 117) | | |
| ORF1ab | | | CLDAGI | CLEASF | HLA- | 56 |
| | | | NYV | NYL | A*02:01 | |
| | | | (SEQ ID | (SEQ ID | | |
| | | | NO: 118) | NO: 119) | | |
| | | | WLMW | WLMWL | HLA- | 67 |
| | | | FIISI | IINL | A*02:01 | |
| | | | (SEQ ID | (SEQ ID | | |
| | | | NO: 120) | NO: 121) | | |
| | | | ILLLDQ | ILLLDQ | HLA- | 89 |
| | | | VLV | ALV | A*02:01 | |
| | | | (SEQ ID | (SEQ ID | | |
| | | | NO: 122) | NO: 123) | | |
| | | | LLCVL | SACVLA | HLA- | 56 |
| | | | AALV | AEC | A*02:01 | |
| | | | (SEQ ID | (SEQ ID | | |
| | | | NO: 124) | NO: 125) | | |
| | | | ALSGV | SLPGVF | HLA- | 78 |
| | | | FCGV | CGV | A*02:01 | |
| | | | (SEQ ID | (SEQ ID | | |
| | | | NO: 126) | NO: 127) | | |
| | | | TLMNV | TLMNV | HLA- | 89 |
| | | | ITLV | LTLV | A*02:01 | |
| | | | (SEQ ID | (SEQ ID | | |
| | | | NO: 128) | NO: 129) | | |
| | | | SMWAL | SMWAL | HLA- | 89 |
| | | | VISV | IISV | A*02:01 | |
| | | | (SEQ ID | (SEQ ID | | |
| | | | NO: 130) | NO: 131) | | |
| ORF6 | | | MFHLV | | | 68 |
| | | | DFQVTI | | | |
| | | | AEIL | | | |
| | | | (SEQ ID | | | |
| | | | NO: 132) | | | |
| ORF7a | | | VFTSCE | | | 85 |
| | | | LYHYQ | | | |
| | | | ECVR | | | |
| | | | (SEQ ID | | | |
| | | | NO: 133) | | | |
| | | | ELYHY | | | |
| | | | QECVR | | | |
| | | | GTTVL | | | |
| | | | (SEQ ID | | | |
| | | | NO: 134) | | | |
| | | | QECVR | | | |
| | | | GTTVL | | | |
| | | | LKEPC | | | |
| | | | (SEQ ID | | | |
| | | | NO: 135) | | | |
| | | | PSGTYE | | | |
| | | | GNSPFH | | | |
| | | | PLA | | | |
| | | | (SEQ ID | | | |
| | | | NO: 136) | | | |
| | | | EGNSPF | | | |
| | | | HPLAD | | | |
| | | | NKFA | | | |
| | | | (SEQ ID | | | |
| | | | NO: 137) | | | |
| | | | FHPLA | | | |
| | | | DNKFA | | | |
| | | | LTCTS | | | |
| | | | (SEQ ID | | | |
| | | | NO: 138) | | | |
| | | | SVSPKL | | | |
| | | | FIRQEE | | | |
| | | | VQQ | | | |
| | | | (SEQ ID | | | |
| | | | NO: 139) | | | |
| | | | LFIRQE | | | |
| | | | EVQQE | | | |
| | | | LYSP | | | |
| | | | (SEQ ID | | | |
| | | | NO: 140) | | | |
| REP | | | | TTDPSF | HLA- | |
| | | | | LGRY | A*01:01 | |
| | | | | (SEQ ID | | |
| | | | | NO: 141) | | |
| | | | | PTDNYI | HLA- | |
| | | | | TTY | A*01:01 | |
| | | | | (SEQ ID | | |
| | | | | NO: 142) | | |

N-Nucleocapsid Protein;
S-Spike Protein;
M-Membrane protein;
E-Envelope protein;
ORF-open reading frame (predicted by similarity)
and
REP (Replicase polyprotein lab).

### RESULTS

### NP Preparation and Physico-Chemical Characterization

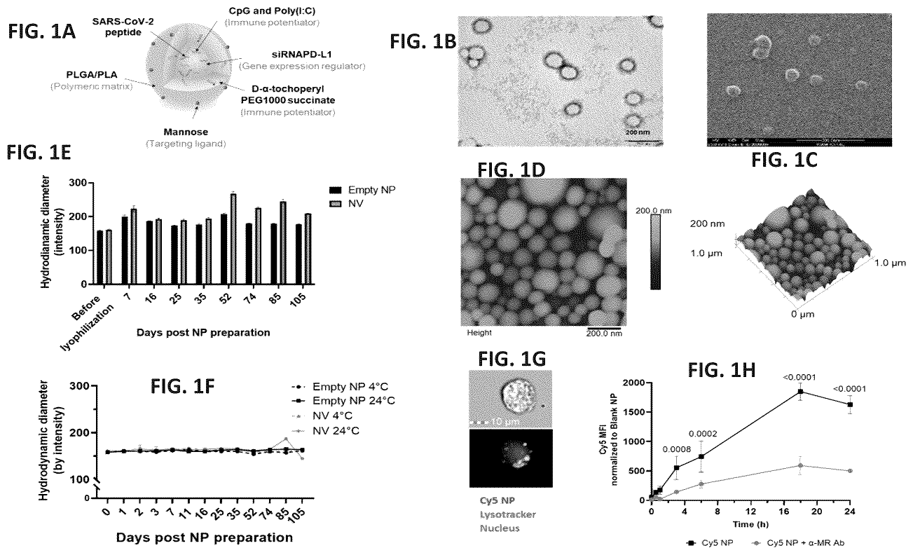

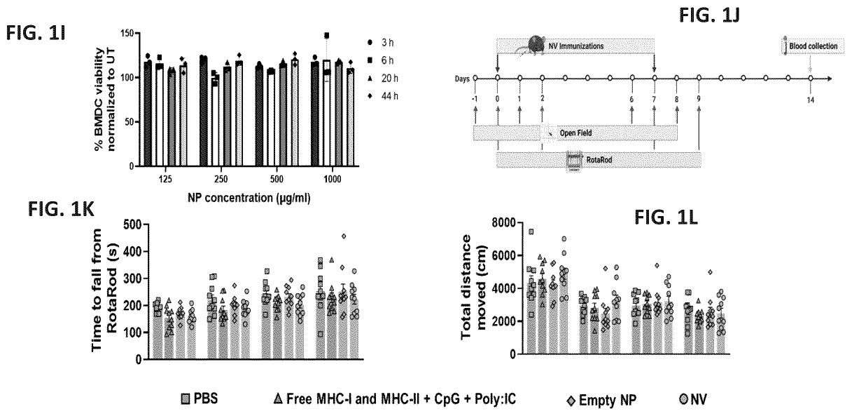

A double emulsion solvent evaporation method was used to develop several nanovaccine (NV) candidates, containing PLGA/PLA NP incorporating the SARS-CoV-2 antigen sequences (MHC-I or MHC-II) as detailed in Table 1, together with Poly(I:C) and CpG oligodeoxynucleotides, which are TLR3 and TLR9 agonists, respectively, as illustrated in FIG. 1A. An siRNA targeting the PD-L1 expression on DC was also co-entrapped with the TLR adjuvants and the SARS-CoV-2 antigens with highest immunogenicity (SEQ ID NOs: 14 and 15), as an attempt to regulate the PD-L1/PD-1 signaling within the DC-T cell interface.

The NP's hydrodynamic diameters ranged between 165-274 nm, with low polydispersity index ( [symbol] ), depending on the incorporated peptides. Electron microscopy and atomic force microscopy (AFM) demonstrated uniform spherical morphology (FIGS. 1B-C) with a slightly rough surface (FIG. 1D). NV presented entrapment efficiency (EE) and loading capacity (LC) ranging from 54.7±1.4% and 27.4±0.7 μg/mg to 99.5±0.1% and 99.5±0.1 μg/mg, respectively for the MHC-I-restricted peptide antigens. Similar values were obtained for peptide sequences predicted as MHC-II ligands, and high levels of EE were also quantified for the distinct oligonucleotide-based immune modulators (CpG, Poly (I:C) and siRNA targeting PD-L1.

Empty NP and NV formulations (containing MHC-I or MHC-II peptides, and TLR ligands) were lyophilized to further analyze the suitability of this formulation to be stored as powder at distinct temperatures. To this end, NP mean diameters, polydispersity index and zeta potential was evaluated at different time-points after lyophilization (using 5% trehalose as a cryoprotectant) (FIG. 1E). The physico-chemical properties of NP and NV stored as suspensions in PBS were also assessed at 4° C. or 24° C., over 3.5 months (FIG. 1F). NP's physicochemical properties remained close to the target specification (200 nm, [symbol] <0.2, neutral surface charge) over time, as a powder or a suspension, at both temperatures (FIG. 1E, F). These data support the potential role of this NV formulation to be widely distributed to low- and middle-income countries thus contributing to increase the current very low vaccination rates, and thereby help to contain the circulation of high levels of the virus worldwide.

Cy5-grafted PLGA polymer was used to prepare fluorescent NP, which enabled the study of NP internalization profile by primary murine bone marrow-derived dendritic cells (BMDC), by ImageStream and Fluorescence-activated cell sorting (FACS). NP were internalized by these APC and trafficked along the endocytic pathway after a 1 h-incubation (FIG. 1G). The Cy5 intensity detected in BMDC was significantly decreased when the mannose receptor was blocked by an α-MR antibody (FIG. 1H). These results confirm that one of the NP internalization pathways is mediated via MR/CD206 receptor on DC surface.

### Preclinical Safety

To evaluate the physiological biocompatibility and in vivo safety of the NP formulation, the present inventors performed an in vitro viability assay, in vivo behavioral assays, and monitored blood chemistry and complete blood count (CBC) following the subcutaneous (s.c.) administration of two doses of NP or NV in naïve mice, one week apart (FIG. 1J). The BMDC viability was evaluated by XTT, following NP incubation in serial concentrations (125, 250, 500, and 1000 μg/ml) over 44 h. The NP did not change BMDC viability at the concentration range tested (FIG. 1I), over time, which supports their physiological biocompatibility.

NP and NV did not cause significant changes in kidney and liver functions, as shown by blood chemistry analysis, nor significant changes in blood count. Moreover, this nanoparticulate-based vaccine did not change motor coordination, imbalance, learning and neurotoxicity in RotaRod studies (rotating rod at increasing velocity)—FIGS. 1K-L.

### Immunization with NV Triggered Cellular and Humoral Responses Against SARS-CoV-2

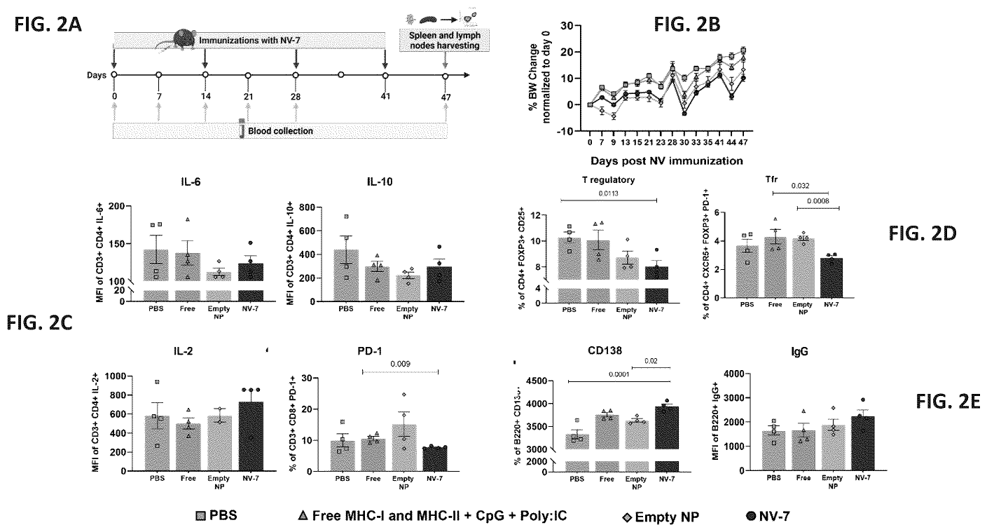

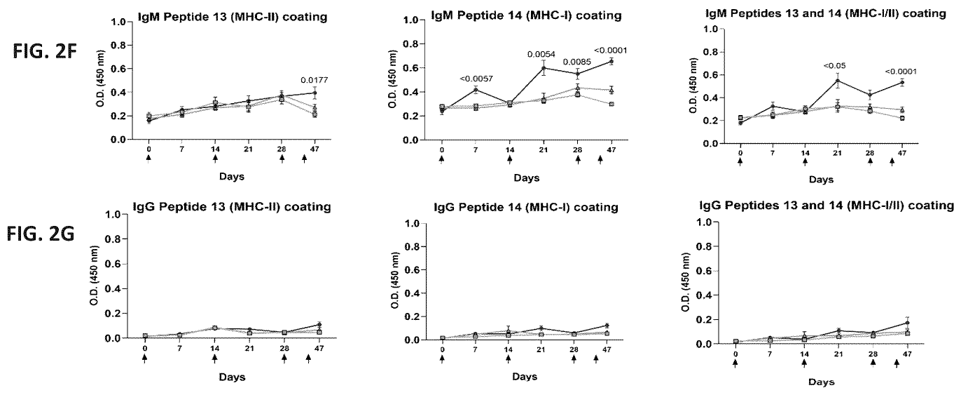

The peptide pairs, combined with TLR agonists CpG and poly (I:C) were tested in vivo to characterize the cellular and humoral responses induced in naïve mice. Three doses of the first seven vaccine candidates (NV-1-7) administered individually (NV1-7) or in combination (NV-5-7) one weck apart induced moderate cellular and humoral responses. Next, NV-7 (SEQ ID NOS: 13 & 14) was administered four times, two weeks apart (FIGS. 2A-B). An increase in the levels of Interleukin-2 (IL-2) in splenic CD4⁺ T cells (FIG. 2C) together with the significant lower expression of programmed cell death protein 1 (PD-1) by CD8⁺ T cells (FIG. 2C) quantified in this organ one week after the last dose (FIG. 2A) was noted.

In addition, NV-7-vaccinated mice also presented lower levels of splenic T follicular regulatory cells (Tfr) compared to controls (FIG. 2D) which is known to correlate with increased antibody secretion. An increase of memory B cells (IgG⁺ B lymphocytes) and plasma cells (CD138⁺ B lymphocytes) in the inguinal lymph nodes was observed in the group treated with our NV-7 (FIG. 2E), which corroborates the NV on Tfr cell function, and supports the potential ability of NV to overcome SARS-CoV-2 infection in the future, as these memory B cells remain for several years and can rapidly differentiate into high-affinity antibody-secreting cells in case this specific antigen will be re-encountered.

To further characterize the additional effect of NV-7 on the secretion of antibodies that specifically bind to SARS-CoV-2 immunogen peptide and RBD protein, blood serum from the vaccinated mice was collected and an ELISA was performed to detect SARS-CoV-2-specific IgM and IgG levels (FIGS. 2F-G). NV-7 increased the secretion of IgM antibodies with reactivity mainly against the Peptide SEQ ID NO: 14, which peaked 7 days after each of the NV-7 doses. Therefore, it was hypothesized that the humoral response elicited by NV-7 was mainly attributed to the MHC-I-restricted peptide SEQ ID NO: 14-loaded NP. An in vitro cell study showed that these antibodies were able to block the entry of Spike-expressing Virus-like particles (VLPs) in 293T cells genetically engineered to stably express human ACE2. Therefore, NV-7 successfully induced the secretion of antibodies with the potential to neutralize SARS-CoV-2 entry into cells.

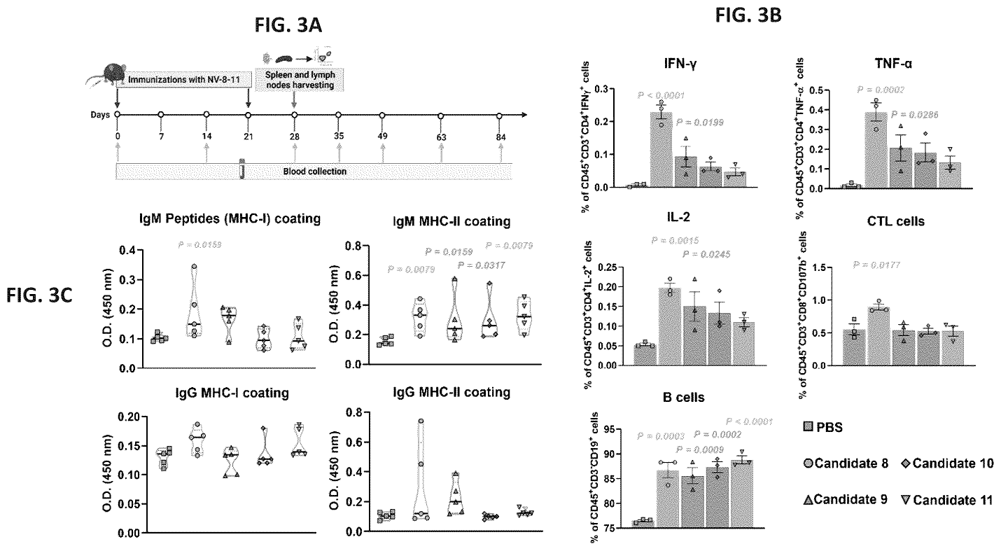

The humoral and cellular responses induced by each of the vaccine candidates (NV8-11) were evaluated following a two-dose, injected 21 days apart vaccination schedule (FIG. 3A) (similar to the schedule use for FDA approved COVID-19 vaccines). On day 28, a week after the second immunization, a significant Th1 and Th2 cytokine secretion was observed, supported by high levels of IFN-γ. TNFα and IL-2 (Th1-guided response) secreted by the splenocytes of mice immunized with NV-8 (FIG. 3B). Moreover, NV-8 was the only NV formulation that harnessed the induction of CD8⁺ T-cell responses (FIG. 3B), which demonstrates the great potential of this formulation to mount a robust adaptive immunity. Furthermore, IgG peak levels induced by NV-8 were reached on day 35 (two weeks after boosting) against entrapped RBD peptide ligands, while RBD MHC-I peptide IgM levels peaked at day 28, and then begin to decline, as expected (FIG. 3C). Importantly, no differences were observed between the body weight of animals immunized with each of the NV formulations and the control group at any time point.

Animals were vaccinated with NV-8 on day 0 and day 21 with SARS-CoV-2 peptides in solution with adjuvants (CpG, and Poly (I:C)) on day 95 of the experiment (FIG. 4E). On day 28, the administration of the same antigens and adjuvants in solution did not induce significant effector CD4⁺ and CD8⁺ T-cell responses, as the CD4⁺ Th1- and CD8⁺ T-cell populations failed to differentiate into IFN-γ, TNF-α and IL-2 producers (FIG. 4B). Importantly, NP empty did not elicit cellular responses, nor developed antibodies (IgM and IgG) against RBD protein and peptide antigens, which were significantly increased in the NV-8 group compared with animals vaccinated with immunogens in solution (FIG. 4C). In addition, the RBD peptide antigen-specific T cell responses were further confirmed using enzyme linked immuno spot (ELISpot) assay that showed the highest overall IFN-γ production by splenocytes of NV-8 mice upon stimulation with MHC-I and MHC-II-restricted RBD peptides (FIG. 4D). Vaccinated animals were challenged with SARS-CoV-2 peptides in solution with adjuvants (CpG, and Poly (I:C)) on day 95 of the experiment (FIG. 4E). The SARS-CoV-2 peptides and adjuvants were injected i.v. to mimic the circulating viral fractions in the blood following SARS-CoV-2 infection, as this combination can stimulate a similar immune response via TLR. The specific SARS-CoV-2 antibodies maintained their high level in the vaccinated mice sera 3 weeks after the second vaccination, afterwards, the antibodies level decayed and on day 91 the antibodies level in the vaccinated group was similar to the control groups. Following the challenge treatment, the specific SRAS-CoV-2 antibodies level increased exclusively in the group of the mice that were treated with NV-8, compared to the control groups (FIG. 4F). In addition, we showed elevation in the levels of effector memory T cells (CD4+ and CD8+) and in memory B cells in the NV-8 group compared to the control groups (FIG. 4G). Importantly, cytokine secretion (e.g. TNF-α and IFN-γ) in the NV-8 group was lower than the PBS and Free groups, which can indicate a better prognosis in case of a viral infection, as high levels of cytokines were found to be associated with severe COVID-19 patients.

### Co-Delivery of SARS-CoV-2 Peptide Antigens, TLR Ligands and siRNA Targeting PD-L1 (siPD-L1) Elicited Strong Antibody Immunity

Polyarginine polymer-siPD-L1 complexes were added to the previous NV-8 formulation. The ability of siPD-L1-loaded NV-8 to in vivo target and down-regulate the expression of this immune checkpoint was confirmed by a significant decrease of 50% PD-L1 and 50% PD-1 mRNA levels quantified in splenocytes of animals immunized with siPD-L1 NV-8 when compared to negative control of scrambled siRNA (siNC)-loaded NV-8 to mice (FIG. 5A). The immunogenicity of siPD-L1-loaded NV-8 was analyzed in vivo using the same immunization schedule of previous studies (FIG. 5B). Interestingly, a significant reduction on PD-1 and PD-L1 mRNA levels was also observed in spleenocytes of siPD-L1 NV-8 immunized animals, one week after a booster dose (FIG. 5C). Moreover, these animals also presented a significant increase on germinal center B cell proliferation (FIG. 5D). As germinal centers are the site of antibody diversification and affinity maturation, such results pointed to a robust antibody development. In fact, animals immunized with siPD-L1 NV-8 presented the highest levels of total IgG against SARS-CoV-2 peptides (Peptide SEQ ID NOs: 14 and 15) and RBD wild-type (RBD-WT) at day 35 (FIG. 5E). Additionally, predominant IgG1 and IgG2a subclass levels against MHC-I and MHC-II-restricted epitopes corroborate the stronger immunogenicity of this multiepitope siRNA vaccine (FIG. 5F). This data in indeed particularly important as the IgG1 is the key T_{H}2-associated isotype antibody and IgG2a has a much stronger Fcγ Receptor (FcγR)-mediated activity, providing the bridge between antigen and FcγR, being therefore well suited to drive viral clearance via antibody-dependent cellular cytotoxicity. A surrogate virus neutralization test (sVNT) (FIG. 5G) was subsequently used to quantify the antibodies able to bind, but also neutralize SARS-CoV-2 RBD-WT and RBD variants. Purified RBD-WT and variants (alfa, beta, delta, gamma, and omicron) and the host cell receptor ACE2, mimic the virus-host interaction in an ELISA plate well. This RBD-ACE2 interaction is blocked (neutralized) using animal sera, in the same manner as in live virus conventional virus neutralization test (cVNT). Differences on neutralizing antibodies (NAbs) were quantified in the serum of animals vaccinated with NV formulations, with or without siRNA, despite presenting comparable RBD IgG titers (FIGS. 5H, J). These data indicate that the NAbs on animals immunized with the NV (NV-8, siNC NV-8 or siPD-L1 NV-8) were able to effectively block RBD-ACE2 interaction, in contrast to the animals immunized with the same antigens, adjuvants, and siRNA in solution (FIG. 5I). We observed a slightly decrease on the ability of our NV to block RBD-ACE2 interaction on RBD variants, however, the NAbs induced by siPD-L1 NV-8 were the most effective on blocking RBD-ACE2 interaction among all known RBD variants, including omicron (FIG. 5I).

### NV Intranasal Boost Elicited a Strong CD4 and CD8 Cellular Response

The nasal administration of a COVID-19 vaccine is a promising strategy to block the transmission of SARS-CoV-2, in addition to preventing the severe form of this disease, following its intramuscular administration. To evaluate the potential of NV for mucosal vaccination, mice were boosted intranasally 3 weeks after the administration of NV subcutaneously (FIG. 6A). One week after, spleen and lymph nodes were collected, and the cellular response characterized. The Bronchoalveolar lavage Fluid (BALF) was also collected to quantify the levels of secretory IgA at the pulmonary mucosa following the nasal administration of the NV-8 booster. FIG. 6B shows that NV elicited a strong CD4 when delivering SARS-CoV-2 peptide, TLR ligands and siRNA targeting PD-L1 secretion. A significant increase on IFN-γ-CD8⁺T cells was found for NV delivering the combinations of SARS-CoV-2 peptides and TLR agonists, which was significantly higher than the one quantified in animals immunized with the mRNA vaccine. FIG. 6C shows significant higher levels of secretory IgA quantified in the BALF of animals immunized with NV SARS-CoV-2 peptide, TLR ligands and siRNA targeting PD-L1 secretion. Increased levels of systemic IgA were also quantified in the serum of these animals when compared to that received prime and booster doses subcutaneously.

### Conclusions

The polymeric nanoparticles (NP)-based vaccine allows the concomitant delivery to DC of selected SARS-CoV-2 antigens with modulators of immune cell function, thus promoting their accumulation at specific cellular and subcellular sites, and subsequently reprogramming immune cell phenotype and genetic properties. The present inventors show that the disclosed nanoparticles offer the potential to overcome biological barriers, while being able to stabilize long but also short peptides as infectious diseases-associated antigens with high loadings—no need for protein translation, processing—being therefore ready for conjugation to MHC proteins, which results in an extensive antigen presentation. The nanoparticles can entrap RNAi polyplex, without compromising nano-carrier physicochemical properties, enabling in vivo siRNA effect on dendritic cells (e.g. reduction of the secretion of TGF-β or PD-L1).

The nanoparticles described herein are able to upregulate DC activation and expansion, cytokine release and subsequent induction of T and B cell immunity, including memory B cells to trigger a fast re-activation of immune response once the specific antigen is re-encountered. It was found that this vaccine increased the antibody production following parenteral, but also nasal immunization, and the antigen-specific nature of the induced anti-tumor cellular immunity was confirmed to be stronger while co-delivering a siRNA targeting the secretion of the immunosuppressive cytokine PD-L1 by DC. It thus counter reacted the expansion and activation of regulatory T cells (Treg), making this also relevant to trigger a protective immunity against SARS-CoV-2 as previously shown to have a role in SARS-COV.

The presently disclosed nanoparticles makes use of 3 complementary mechanisms of action against SARS-CoV-2 infection. The present data indicates that it (1) induces the production of high levels of antibodies that are able to recognize and bind to the correspondent sequence at the virus, thus blocking, for example, the Spike protein (while delivering Spike antigens) that the virus uses to infect the cells. At the same time, it improves the (2) activation of T cells that then recognize and kill cells infected with SARS-CoV-2 virus. Moreover, the vaccine was also shown to (3) modulate T follicular helper (Tfh) cells, driving a B cell isotype switching and affinity maturation, thus indicating a potential production of high-affinity antibodies. Therefore, this manipulation of follicular T cell development using the disclosed nanoparticles will be particularly important to guarantee protection for vulnerable groups of patients with decaying immune function, such as the elderly population and individuals suffering from debilitating chronic diseases, for whom the protection afforded by vaccination is crucial but still sub-optimal. This high-risk population in fact accounts for most of the severe disease and fatalities due to COVID-19.

The disclosed SARS-CoV-2 vaccine enables the protection of individuals not yet infected by increasing the production of SARS-CoV-2 specific binding and neutralizing antibodies, but also allows the destruction of infected cells by triggering an extensive but specific cellular immunity. Moreover, the activation of the local immunity at mucosal surfaces (oral, sublingual, nasal, ocular, buccal, rectal and vaginal) triggered by the nanoparticles indicates that the nanoparticles have the potential to be used in the treatment/prevention of COVID-19.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, the priority document of this application is hereby incorporated herein by reference in its entirety.

1. A polymeric nanoparticle comprising:
(i) at least one SARS-CoV-2 derived antigen which is capable of producing a B-cell and/or a T-cell response; and
(ii) an antigen presenting cell targeting moiety which is attached to the outer surface of the nanoparticle.

2. (canceled)

3. The polymeric nanoparticle of claim 1, further comprising a polynucleotide agent capable of downregulating an amount of a polypeptide in said dendritic cell, wherein the polynucleotide agent is entrapped in the nanoparticle.

4. The polymeric nanoparticle of claim 1, further comprising d-α-tocopheryl polyethylene glycol 1000 succinate (TPGS).

5. The polymeric nanoparticle of claim 1, further comprising at least one toll-like receptor ligand which is entrapped in the nanoparticle.

6. (canceled)

7. The polymeric particle of claim 1, further comprising at least one adjuvant.

8-18. (canceled)

19. The polymeric nanoparticle of claim 1, wherein said at least one SARS-CoV-2 derived antigen comprises the amino acid sequence selected from the group consisting of SEQ ID NOs: 1-142.

20. The polymeric nanoparticle of claim 1, wherein said at least one SARS-CoV-2 derived antigen comprises the amino acid sequence selected from the group consisting of SEQ ID NOs: 1-21.

21. The polymeric nanoparticle of claim 1, wherein said at least one SARS-CoV-2 derived antigen comprises the amino acid sequence as set forth in SEQ ID NO: 14 and/or SEQ ID NO: 15.

22-25. (canceled)

26. The polymeric nanoparticle of claim 1, being fabricated from PLGA and PLA, and optionally further comprising PVA.

27-28. (canceled)

29. The polymeric nanoparticle of claim 7, wherein said at least one adjuvant comprises a Toll-like receptor (TLR) ligand.

30-31. (canceled)

32. The polymeric nanoparticle of claim 7, wherein said at least one adjuvant comprises a retinoic-acid-inducible protein 1 (RIG-I)-like receptor ligand.

33-34. (canceled)

35. The polymeric nanoparticle of claim 7, wherein said at least one adjuvant is selected from the group consisting of hyaluronic acid (HA), poloxamer 407, 2′,3′-cGAMP, chitosan, Dectin-1 agonist laminarin and β-glucan.

36. The polymeric nanoparticle of claim 1, further comprising a surfactant selected from the group consisting of d-α-tocopheryl polyethylene glycol 1000 succinate (TPGS), poly(vinyl alcohol) (PVA) and poloxamer 407.

37. (canceled)

38. The polymeric nanoparticle of claim 1, further comprising a polynucleotide agent capable of downregulating an amount of a polypeptide in said dendritic cell.

39. The polymeric nanoparticle of claim 38, wherein said polynucleotide agent is selected from the group consisting of an antisense polynucleotide, siRNA, gRNA, miRNA, a DNAzyme and a Ribozyme.

40. The polymeric nanoparticle of claim 39, wherein said polypeptide is selected from the group consisting of SARS-CoV-2 Spike glycoprotein, Membrane glycoproteins (region 220-241), Nucleocapsid and envelope proteins, SARS Replicase and RNA Polymerase region, TGF-β, VEGFA, PD-L1/PD-1, VEGFR1, VEGFR2, VEGFR3, IDO, RANKL, IL-10, IL-6/IL-6R, IL-1, IL-28A, IL-28B, IL-29, IP-10/CXCL10 (interferon γ-inducible protein 10), CD16, ITAM (immunoreceptor tyrosine-based activation motif), DC-SIGN (dendritic cell specific intercellular adhesion molecule-grabbing nonintegrin), ICAM-3 (intercellular adhesion molecule 3) and PGE2 receptor.

41. The polymeric nanoparticle of claim 39, wherein said polynucleotide agent is siRNA.

42. The polymeric nanoparticle of claim 41, wherein said siRNA is complexed with a polymer.

43. The polymeric nanoparticle of claim 42, wherein said polymer is selected from the group consisting of glutamate chitosan, poly-arginine, alkylated poly(α)glutamate amine (APA) and poly-(α)glutamic acid (PGA).

44. The polymeric nanoparticle of claim 1, wherein said antigen presenting cell targeting moiety is selected from the group consisting of mannose, tri-mannose, PEG-mannose, laminarin and PEG-laminarin.

45. (canceled)

46. A method of treating or preventing COVID-19 in a subject in need thereof comprising administering to the subject a therapeutically effective amount of the polymeric nanoparticles of claim 1, thereby treating or preventing COVID-19.

47. (canceled)

48. The method of claim 46, wherein said administering is subcutaneous, intradermal, intramuscular, intravenous or mucosal.

49. The method of claim 48, wherein said mucosal is nasal.


SEQUENCE LISTING (149 sequences)

SEQ ID NO: 1 - protein, 9 aa, Artificial sequence
  short peptide sequence derived from SARS-CoV-2
VRFPNITNL

SEQ ID NO: 2 - protein, 18 aa, Artificial sequence
  short peptide sequence derived from SARS-CoV-2
FSQILPDPSKPSKRSFIE

SEQ ID NO: 3 - protein, 10 aa, Artificial sequence
  short peptide sequence derived from SARS-CoV-2
MEVTPSGTWL

SEQ ID NO: 4 - protein, 15 aa, Artificial sequence
  short peptide sequence derived from SARS-CoV-2
PKGFYAEGSRGGSQA

SEQ ID NO: 5 - protein, 9 aa, Artificial sequence
  short peptide sequence derived from SARS-CoV-2
LITGRLQSL

SEQ ID NO: 6 - protein, 14 aa, Artificial sequence
  short peptide sequence derived from SARS-CoV-2
GAALQIPFAMQMAY

SEQ ID NO: 7 - protein, 10 aa, Artificial sequence
  short peptide sequence derived from SARS-CoV-2
DLLFNKVTLA

SEQ ID NO: 8 - protein, 15 aa, Artificial sequence
  short peptide sequence derived from SARS-CoV-2
DLCFTNVYADSFVIR

SEQ ID NO: 9 - protein, 10 aa, Artificial sequence
  short peptide sequence derived from SARS-CoV-2
GGDGKMKDLS

SEQ ID NO: 10 - protein, 15 aa, Artificial sequence
  short peptide sequence derived from SARS-CoV-2
AIVLQLPQGTTLPKG

SEQ ID NO: 11 - protein, 9 aa, Artificial sequence
  short peptide sequence derived from SARS-CoV-2
RYRIGNYKL

SEQ ID NO: 12 - protein, 15 aa, Artificial sequence
  short peptide sequence derived from SARS-CoV-2
LSYYKLGASQRVAGD

SEQ ID NO: 13 - protein, 15 aa, Artificial sequence
  short peptide sequence derived from SARS-CoV-2
FPLQSYGFQPTNGVG

SEQ ID NO: 14 - protein, 9 aa, Artificial sequence
  short peptide sequence derived from SARS-CoV-2
YRLFRKSNL

SEQ ID NO: 15 - protein, 20 aa, Artificial sequence
  short peptide sequence derived from SARS-CoV-2
NGVEGFNCYFPLQSYGFQPT

SEQ ID NO: 16 - protein, 10 aa, Artificial sequence
  short peptide sequence derived from SARS-CoV-2
PLQSYGFQPT

SEQ ID NO: 17 - protein, 10 aa, Artificial sequence
  short peptide sequence derived from SARS-CoV-2
NGVEGFNCYF

SEQ ID NO: 18 - protein, 10 aa, Artificial sequence
  short peptide sequence derived from SARS-CoV-2
EGFNCYFPLQ

SEQ ID NO: 19 - protein, 9 aa, Artificial sequence
  T cell epitope amino acid sequence
SKVGGNYNY

SEQ ID NO: 20 - protein, 18 aa, Artificial sequence
  MHC class II epitope amino acid sequence
PKGFYAEGSRGGSQASSR

SEQ ID NO: 21 - protein, 16 aa, Artificial sequence
  MHC class I epitope amino acid sequence
GAALQIPFAMQMAYRF

SEQ ID NO: 22 - protein, 9 aa, Artificial sequence
  SARS-CoV-I and 2 partial peptide sequence
ALNTPKDHI

SEQ ID NO: 23 - protein, 9 aa, Artificial sequence
  SARS-CoV-I and 2 partial peptide sequence
LALLLLDRL

SEQ ID NO: 24 - protein, 9 aa, Artificial sequence
  SARS-CoV-I and 2 partial peptide sequence
LLLDRLNQL

SEQ ID NO: 25 - protein, 9 aa, Artificial sequence
  SARS-CoV-I and 2 partial peptide sequence
RLNQLESKV

SEQ ID NO: 26 - protein, 9 aa, Artificial sequence
  SARS-CoV-I and 2 partial peptide sequence
RLNQLESKM

SEQ ID NO: 27 - protein, 10 aa, Artificial sequence
  SARS-CoV-I and 2 partial peptide sequence
GETALALLLL

SEQ ID NO: 28 - protein, 10 aa, Artificial sequence
  SARS-CoV-I and 2 partial peptide sequence
GDAALALLLL

SEQ ID NO: 29 - protein, 10 aa, Artificial sequence
  SARS-CoV-I and 2 partial peptide sequence
MEVTPSGTWL

SEQ ID NO: 30 - protein, 48 aa, Artificial sequence
  SARS-CoV-I and 2 partial peptide sequence
KHIDAYKTFPPTEPKKDKKKKTDEAQPLPQRQKKQPTVTLLPAADMDD

SEQ ID NO: 31 - protein, 47 aa, Artificial sequence
  SARS-CoV-I and 2 partial peptide sequence
NKHIDAYKTFPPTEPKKDKKKKTDEAQPLPQRQKKQPTVTLLPAADM

SEQ ID NO: 32 - protein, 21 aa, Artificial sequence
  SARS-CoV-I and 2 partial peptide sequence
NNAATVLQLPQGTTLPKGFYA

SEQ ID NO: 33 - protein, 20 aa, Artificial sequence
  SARS-CoV-I and 2 partial peptide sequence
NNNAATVLQLPQGTTLPKGF

SEQ ID NO: 34 - protein, 15 aa, Artificial sequence
  SARS-CoV-I and 2 partial peptide sequence
DAALALLLLDRLNQL

SEQ ID NO: 35 - protein, 17 aa, Artificial sequence
  SARS-CoV-I and 2 partial peptide sequence
ALALLLLDRLNQLESKV

SEQ ID NO: 36 - protein, 15 aa, Artificial sequence
  SARS-CoV-I and 2 partial peptide sequence
MSRIGMEVTPSGTWL

SEQ ID NO: 37 - protein, 17 aa, Artificial sequence
  SARS-CoV-I and 2 partial peptide sequence
MEVTPSGTWLTYHGAIK

SEQ ID NO: 38 - protein, 9 aa, Artificial sequence
  SARS-CoV-I and 2 partial peptide sequence
PINTNSSPD

SEQ ID NO: 39 - protein, 9 aa, Artificial sequence
  SARS-CoV-I and 2 partial peptide sequence
PINTNSGPD

SEQ ID NO: 40 - protein, 18 aa, Artificial sequence
  SARS-CoV-I and 2 partial peptide sequence
LSPRWYFYYLGTGPEASL

SEQ ID NO: 41 - protein, 15 aa, Artificial sequence
  SARS-CoV-I and 2 partial peptide sequence
FYYLGTGPEASLPYG

SEQ ID NO: 42 - protein, 15 aa, Artificial sequence
  SARS-CoV-I and 2 partial peptide sequence
SPRWYFYYLGTGPEA

SEQ ID NO: 43 - protein, 13 aa, Artificial sequence
  SARS-CoV-I and 2 partial peptide sequence
YFYYLGTGPEASL

SEQ ID NO: 44 - protein, 9 aa, Artificial sequence
  SARS-CoV-I and 2 partial peptide sequence
QFAPSASAF

SEQ ID NO: 45 - protein, 17 aa, Artificial sequence
  SARS-CoV-I and 2 partial peptide sequence
KHWPQIAQFAPSASAFF

SEQ ID NO: 46 - protein, 17 aa, Artificial sequence
  SARS-CoV-I and 2 partial peptide sequence
KHWPQIAQFAPSASAFF

SEQ ID NO: 47 - protein, 15 aa, Artificial sequence
  SARS-CoV-I and 2 partial peptide sequence
AQFAPSASAFFGMSR

SEQ ID NO: 48 - protein, 18 aa, Artificial sequence
  SARS-CoV-I and 2 partial peptide sequence
AQFAPSASAFFGMSRIGM

SEQ ID NO: 49 - protein, 15 aa, Artificial sequence
  SARS-CoV-I and 2 partial peptide sequence
AIVLQLPQGTTLPKG

SEQ ID NO: 50 - protein, 22 aa, Artificial sequence
  SARS-CoV-I and 2 partial peptide sequence
FGAGAALQIPFAMQMAYRFNGI

SEQ ID NO: 51 - protein, 16 aa, Artificial sequence
  SARS-CoV-I and 2 partial peptide sequence
GAALQIPFAMQMAYRF

SEQ ID NO: 52 - protein, 18 aa, Artificial sequence
  SARS-CoV-I and 2 partial peptide sequence
GAGAALQIPFAMQMAYRF

SEQ ID NO: 53 - protein, 9 aa, Artificial sequence
  SARS-CoV-I and 2 partial peptide sequence
FIAGLIAIV

SEQ ID NO: 54 - protein, 9 aa, Artificial sequence
  SARS-CoV-I and 2 partial peptide sequence
LITGRLQSL

SEQ ID NO: 55 - protein, 9 aa, Artificial sequence
  SARS-CoV-I and 2 partial peptide sequence
NLNESLIDL

SEQ ID NO: 56 - protein, 9 aa, Artificial sequence
  SARS-CoV-I and 2 partial peptide sequence
ALNTLVKQL

SEQ ID NO: 57 - protein, 9 aa, Artificial sequence
  SARS-CoV-I and 2 partial peptide sequence
RLNEVAKNL

SEQ ID NO: 58 - protein, 9 aa, Artificial sequence
  SARS-CoV-I and 2 partial peptide sequence
VLNDILSRL

SEQ ID NO: 59 - protein, 18 aa, Artificial sequence
  SARS-CoV-I and 2 partial peptide sequence
KSFEIDKGIYQTSNFRVV

SEQ ID NO: 60 - protein, 18 aa, Artificial sequence
  SARS-CoV-I and 2 partial peptide sequence
QLIRAAEIRASANLAATK

SEQ ID NO: 61 - protein, 9 aa, Artificial sequence
  SARS-CoV-I and 2 partial peptide sequence
YLQPRTFLL

SEQ ID NO: 62 - protein, 15 aa, Artificial sequence
  SARS-CoV-I and 2 partial peptide sequence
GAISSVLNDILSRLD

SEQ ID NO: 63 - protein, 17 aa, Artificial sequence
  SARS-CoV-I and 2 partial peptide sequence
AISSVLNDILSRLDKVE

SEQ ID NO: 64 - protein, 15 aa, Artificial sequence
  SARS-CoV-I and 2 partial peptide sequence
IDRLITGRLQSLQTY

SEQ ID NO: 65 - protein, 9 aa, Artificial sequence
  SARS-CoV-I and 2 partial peptide sequence
LITGRLQSL

SEQ ID NO: 66 - protein, 17 aa, Artificial sequence
  SARS-CoV-I and 2 partial peptide sequence
RLITGRLQSLQTYVTQQ

SEQ ID NO: 67 - protein, 9 aa, Artificial sequence
  SARS-CoV-I and 2 partial peptide sequence
RLQSLQTYV

SEQ ID NO: 68 - protein, 17 aa, Artificial sequence
  SARS-CoV-I and 2 partial peptide sequence
EAEVQIDRLITGRLQSL

SEQ ID NO: 69 - protein, 17 aa, Artificial sequence
  SARS-CoV-I and 2 partial peptide sequence
PWYVWLGFIAGLIAIVM

SEQ ID NO: 70 - protein, 15 aa, Artificial sequence
  SARS-CoV-I and 2 partial peptide sequence
WLGFIAGLIAIVMVT

SEQ ID NO: 71 - protein, 15 aa, Artificial sequence
  SARS-CoV-I and 2 partial peptide sequence
LGFIAGLIAIVMVTI

SEQ ID NO: 72 - protein, 32 aa, Artificial sequence
  SARS-CoV-I and 2 partial peptide sequence
DAVDCSQNPLAELKCSVKSFEIDKGIYQTSNF

SEQ ID NO: 73 - protein, 31 aa, Artificial sequence
  SARS-CoV-I and 2 partial peptide sequence
DAVDCALDPLSETKCTLKSFTVEKGIYQTSN

SEQ ID NO: 74 - protein, 20 aa, Artificial sequence
  SARS-CoV-I and 2 partial peptide sequence
ISGINASVVNIQKEIDRLNE

SEQ ID NO: 75 - protein, 10 aa, Artificial sequence
  SARS-CoV-I and 2 partial peptide sequence
YEQYIKWPWY

SEQ ID NO: 76 - protein, 18 aa, Artificial sequence
  SARS-CoV-I and 2 partial peptide sequence
PSKPSKRSFIEDLLFNKV

SEQ ID NO: 77 - protein, 28 aa, Artificial sequence
  SARS-CoV-I and 2 partial peptide sequence
EIDRLNEVAKNLNESLIDLQELGKYEQY

SEQ ID NO: 78 - protein, 20 aa, Artificial sequence
  SARS-CoV-I and 2 partial peptide sequence
NLCPFGEVFNATRFASVYAW

SEQ ID NO: 79 - protein, 31 aa, Artificial sequence
  SARS-CoV-I and 2 partial peptide sequence
ADYSVLYNSASFSTFKCYGVSPTKLNDLCFT

SEQ ID NO: 80 - protein, 15 aa, Artificial sequence
  SARS-CoV-I and 2 partial peptide sequence
VVLSFELLHAPATVC

SEQ ID NO: 81 - protein, 15 aa, Artificial sequence
  SARS-CoV-I and 2 partial peptide sequence
KCYGVSPTKLNDLCF

SEQ ID NO: 82 - protein, 16 aa, Artificial sequence
  SARS-CoV-I and 2 partial peptide sequence
KVGGNYNYLYRLFRKS

SEQ ID NO: 83 - protein, 15 aa, Artificial sequence
  SARS-CoV-I and 2 partial peptide sequence
GGNYNYLYRLFRKSN

SEQ ID NO: 84 - protein, 15 aa, Artificial sequence
  SARS-CoV-I and 2 partial peptide sequence
EVRQIAPGQTGKIAD

SEQ ID NO: 85 - protein, 9 aa, Artificial sequence
  SARS-CoV-I and 2 partial peptide sequence
CVADYSVLY

SEQ ID NO: 86 - protein, 9 aa, Artificial sequence
  SARS-CoV-I and 2 partial peptide sequence
RLFRKSNLK

SEQ ID NO: 87 - protein, 13 aa, Artificial sequence
  SARS-CoV-I and 2 partial peptide sequence
VIRGDEVRQIAPG

SEQ ID NO: 88 - protein, 20 aa, Artificial sequence
  SARS-CoV-I and 2 partial peptide sequence
NVYADSFVIRGDEVRQIAPG

SEQ ID NO: 89 - protein, 13 aa, Artificial sequence
  SARS-CoV-I and 2 partial peptide sequence
VIRGDEVRQIAPG

SEQ ID NO: 90 - protein, 21 aa, Artificial sequence
  SARS-CoV-I and 2 partial peptide sequence
CYFPLQSYGFQPTNGVGYQPY

SEQ ID NO: 91 - protein, 18 aa, Artificial sequence
  SARS-CoV-I and 2 partial peptide sequence
TESNKKFLPFQQFGRDIA

SEQ ID NO: 92 - protein, 9 aa, Artificial sequence
  SARS-CoV-I and 2 partial peptide sequence
NYNYLYRLF

SEQ ID NO: 93 - protein, 24 aa, Artificial sequence
  SARS-CoV-I and 2 partial peptide sequence
MADNGTITVEELKQLLEQWNLVIG

SEQ ID NO: 94 - protein, 24 aa, Artificial sequence
  SARS-CoV-I and 2 partial peptide sequence
MADSNGTITVEELKKLLEQWNLVI

SEQ ID NO: 95 - protein, 21 aa, Artificial sequence
  SARS-CoV-I and 2 partial peptide sequence
PLMESELVIGAVIIRGHLRMA

SEQ ID NO: 96 - protein, 20 aa, Artificial sequence
  SARS-CoV-I and 2 partial peptide sequence
PLLESELVIGAVILRGHLRI

SEQ ID NO: 97 - protein, 9 aa, Artificial sequence
  SARS-CoV-I and 2 partial peptide sequence
KKLLEQWNL

SEQ ID NO: 98 - protein, 9 aa, Artificial sequence
  SARS-CoV-I and 2 partial peptide sequence
KQLLEQWNL

SEQ ID NO: 99 - protein, 15 aa, Artificial sequence
  SARS-CoV-I and 2 partial peptide sequence
GFAAYNRYRIGNYKL

SEQ ID NO: 100 - protein, 15 aa, Artificial sequence
  SARS-CoV-I and 2 partial peptide sequence
NRYRIGNYKLNTDHA

SEQ ID NO: 101 - protein, 23 aa, Artificial sequence
  SARS-CoV-I and 2 partial peptide sequence
DSGFAAYNRYRIGNYKLNTDHAG

SEQ ID NO: 102 - protein, 15 aa, Artificial sequence
  SARS-CoV-I and 2 partial peptide sequence
LSYYKLGASQRVGTD

SEQ ID NO: 103 - protein, 15 aa, Artificial sequence
  SARS-CoV-I and 2 partial peptide sequence
TLSYYKLGASQRVGT

SEQ ID NO: 104 - protein, 16 aa, Artificial sequence
  SARS-CoV-I and 2 partial peptide sequence
TSRTLSYYKLGASQRV

SEQ ID NO: 105 - protein, 10 aa, Artificial sequence
  SARS-CoV-I and 2 partial peptide sequence
TLACFVLAAV

SEQ ID NO: 106 - protein, 9 aa, Artificial sequence
  SARS-CoV-I and 2 partial peptide sequence
GLMWLSYFV

SEQ ID NO: 107 - protein, 9 aa, Artificial sequence
  SARS-CoV-I and 2 partial peptide sequence
GLMWLSYFI

SEQ ID NO: 108 - protein, 9 aa, Artificial sequence
  SARS-CoV-I and 2 partial peptide sequence
HLRMAGHSL

SEQ ID NO: 109 - protein, 9 aa, Artificial sequence
  SARS-CoV-I and 2 partial peptide sequence
HLRIAGHHL

SEQ ID NO: 110 - protein, 15 aa, Artificial sequence
  SARS-CoV-I and 2 partial peptide sequence
MYSFVSEETGTLIVN

SEQ ID NO: 111 - protein, 15 aa, Artificial sequence
  SARS-CoV-I and 2 partial peptide sequence
SVLLFLAFVVFLLVT

SEQ ID NO: 112 - protein, 15 aa, Artificial sequence
  SARS-CoV-I and 2 partial peptide sequence
FLLVTLAILTALRLC

SEQ ID NO: 113 - protein, 15 aa, Artificial sequence
  SARS-CoV-I and 2 partial peptide sequence
ALRLCAYCCNIVNVS

SEQ ID NO: 114 - protein, 15 aa, Artificial sequence
  SARS-CoV-I and 2 partial peptide sequence
RFFTLGSITAQPVKI

SEQ ID NO: 115 - protein, 15 aa, Artificial sequence
  SARS-CoV-I and 2 partial peptide sequence
RIFTIGTVTLKQGEI

SEQ ID NO: 116 - protein, 9 aa, Artificial sequence
  SARS-CoV-I and 2 partial peptide sequence
SITAQPVKI

SEQ ID NO: 117 - protein, 9 aa, Artificial sequence
  SARS-CoV-I and 2 partial peptide sequence
TVTLKQGEI

SEQ ID NO: 118 - protein, 9 aa, Artificial sequence
  SARS-CoV-I and 2 partial peptide sequence
CLDAGINYV

SEQ ID NO: 119 - protein, 9 aa, Artificial sequence
  SARS-CoV-I and 2 partial peptide sequence
CLEASFNYL

SEQ ID NO: 120 - protein, 9 aa, Artificial sequence
  SARS-CoV-I and 2 partial peptide sequence
WLMWFIISI

SEQ ID NO: 121 - protein, 9 aa, Artificial sequence
  SARS-CoV-I and 2 partial peptide sequence
WLMWLIINL

SEQ ID NO: 122 - protein, 9 aa, Artificial sequence
  SARS-CoV-I and 2 partial peptide sequence
ILLLDQVLV

SEQ ID NO: 123 - protein, 9 aa, Artificial sequence
  SARS-CoV-I and 2 partial peptide sequence
ILLLDQALV

SEQ ID NO: 124 - protein, 9 aa, Artificial sequence
  SARS-CoV-I and 2 partial peptide sequence
LLCVLAALV

SEQ ID NO: 125 - protein, 9 aa, Artificial sequence
  SARS-CoV-I and 2 partial peptide sequence
SACVLAAEC

SEQ ID NO: 126 - protein, 9 aa, Artificial sequence
  SARS-CoV-I and 2 partial peptide sequence
ALSGVFCGV

SEQ ID NO: 127 - protein, 9 aa, Artificial sequence
  SARS-CoV-I and 2 partial peptide sequence
SLPGVFCGV

SEQ ID NO: 128 - protein, 9 aa, Artificial sequence
  SARS-CoV-I and 2 partial peptide sequence
TLMNVITLV

SEQ ID NO: 129 - protein, 9 aa, Artificial sequence
  SARS-CoV-I and 2 partial peptide sequence
TLMNVLTLV

SEQ ID NO: 130 - protein, 9 aa, Artificial sequence
  SARS-CoV-I and 2 partial peptide sequence
SMWALVISV

SEQ ID NO: 131 - protein, 9 aa, Artificial sequence
  SARS-CoV-I and 2 partial peptide sequence
SMWALIISV

SEQ ID NO: 132 - protein, 15 aa, Artificial sequence
  SARS-CoV-I and 2 partial peptide sequence
MFHLVDFQVTIAEIL

SEQ ID NO: 133 - protein, 15 aa, Artificial sequence
  SARS-CoV-I and 2 partial peptide sequence
VFTSCELYHYQECVR

SEQ ID NO: 134 - protein, 15 aa, Artificial sequence
  SARS-CoV-I and 2 partial peptide sequence
ELYHYQECVRGTTVL

SEQ ID NO: 135 - protein, 15 aa, Artificial sequence
  SARS-CoV-I and 2 partial peptide sequence
QECVRGTTVLLKEPC

SEQ ID NO: 136 - protein, 15 aa, Artificial sequence
  SARS-CoV-I and 2 partial peptide sequence
PSGTYEGNSPFHPLA

SEQ ID NO: 137 - protein, 15 aa, Artificial sequence
  SARS-CoV-I and 2 partial peptide sequence
EGNSPFHPLADNKFA

SEQ ID NO: 138 - protein, 15 aa, Artificial sequence
  SARS-CoV-I and 2 partial peptide sequence
FHPLADNKFALTCTS

SEQ ID NO: 139 - protein, 15 aa, Artificial sequence
  SARS-CoV-I and 2 partial peptide sequence
SVSPKLFIRQEEVQQ

SEQ ID NO: 140 - protein, 15 aa, Artificial sequence
  SARS-CoV-I and 2 partial peptide sequence
LFIRQEEVQQELYSP

SEQ ID NO: 141 - protein, 10 aa, Artificial sequence
  SARS-CoV-I and 2 partial peptide sequence
TTDPSFLGRY

SEQ ID NO: 142 - protein, 9 aa, Artificial sequence
  SARS-CoV-I and 2 partial peptide sequence
PTDNYITTY

SEQ ID NO: 143 - DNA, 20 bp, Artificial sequence
  CpG-ODN 1826 nucleic acid sequence
tccatgacgttcctgacgtt

SEQ ID NO: 144 - DNA, 23 bp, Artificial sequence
  Single strand DNA oligonucleotide
attctctggttgattttgcggta

SEQ ID NO: 145 - DNA, 23 bp, Artificial sequence
  Single strand DNA oligonucleotide
ttcagatcacagacgtcaagctg

SEQ ID NO: 146 - DNA, 23 bp, Artificial sequence
  Single strand DNA oligonucleotide
ggtgaaaaggacctctcgaagtg

SEQ ID NO: 147 - DNA, 25 bp, Artificial sequence
  Single strand DNA oligonucleotide
atagtcaagggcatatccaacaaca

SEQ ID NO: 148 - DNA, 21 bp, Artificial Sequence
  PD-L1 siRNA
cccacauaaaaaacaguugtt

SEQ ID NO: 149 - protein, 18 aa, Artificial sequence
  SARS-CoV-I and 2 partial peptide sequence
KSFTVEKGIYQTSNFRVQ